SEQUENCE LISTING (39 sequences)

SEQ ID NO: 1 - DNA, 51 bp, homo sapiens
cacagcaaatggccgatcatgtaccatcttgcttcatccacccccaaactg

SEQ ID NO: 2 - DNA, 51 bp, homo sapiens
cacagcaaatggccgatcatgtaccgtcttgcttcatccacccccaaactg

SEQ ID NO: 3 - DNA, 51 bp, homo sapiens
tcatgtttaatatctaactgtatgcgtttatgattaaaaaataaagttgaa

SEQ ID NO: 4 - DNA, 51 bp, homo sapiens
tcatgtttaatatctaactgtatgcttttatgattaaaaaataaagttgaa

SEQ ID NO: 5 - DNA, 52 bp, homo sapiens
tgaactaggatatttggatataagcaatgaactaggatatttggcagaagaa

SEQ ID NO: 6 - DNA, 52 bp, homo sapiens
tgaactaggatatttggatataagcagtgaactaggatatttggcagaagaa

SEQ ID NO: 7 - DNA, 52 bp, homo sapiens
accagccagagagatctgcaggaactatactacagcccgtaagaggtgaggc

SEQ ID NO: 8 - DNA, 52 bp, homo sapiens
accagccagagagatctgcaggaactgtactacagcccgtaagaggtgaggc

SEQ ID NO: 9 - DNA, 51 bp, homo sapiens
tggttaagccagttgttcatacttcctttacaaatataaagatagctgttt

SEQ ID NO: 10 - DNA, 51 bp, homo sapiens
tggttaagccagttgttcatacttcgtttacaaatataaagatagctgttt

SEQ ID NO: 11 - DNA, 33 bp, homo sapiens
agctaggtgtaaagtcagacaagcctgggattg

SEQ ID NO: 12 - DNA, 33 bp, homo sapiens
agctaggtgtaaagtcggacaagcctgggattg

SEQ ID NO: 13 - DNA, 51 bp, homo sapiens
tctaggtcatgacctggagtcccaacgatgaggcagttacccaggtgcctc

SEQ ID NO: 14 - DNA, 51 bp, homo sapiens
tctaggtcatgacctggagtcccaatgatgaggcagttacccaggtgcctc

SEQ ID NO: 15 - DNA, 51 bp, homo sapiens
gcaggcaaataagtaaataaacaaacgaaatcattatagacagcactgggt

SEQ ID NO: 16 - DNA, 51 bp, homo sapiens
gcaggcaaataagtaaataaacaaaggaaatcattatagacagcactgggt

SEQ ID NO: 17 - DNA, 51 bp, homo sapiens
ttatcataattctacataggattagcggagagaaggatctcaaataaatta

SEQ ID NO: 18 - DNA, 51 bp, homo sapiens
ttatcataattctacataggattagtggagagaaggatctcaaataaatta

SEQ ID NO: 19 - DNA, 51 bp, homo sapiens
cagcactttggttcaccttttcttcaatattgtgattctttttgtcataag

SEQ ID NO: 20 - DNA, 51 bp, homo sapiens
cagcactttggttcaccttttcttcgatattgtgattctttttgtcataag

SEQ ID NO: 21 - DNA, 51 bp, homo sapiens
tttcatttgtgctttttctgttcaaatttcttgtatgtgtttcatataaca

SEQ ID NO: 22 - DNA, 51 bp, homo sapiens
tttcatttgtgctttttctgttcaagtttcttgtatgtgtttcatataaca

SEQ ID NO: 23 - DNA, 51 bp, homo sapiens
atgggtcagaacagagtgagtcacacaaaccaccatgcagttcccaggtga

SEQ ID NO: 24 - DNA, 51 bp, homo sapiens
atgggtcagaacagagtgagtcacataaaccaccatgcagttcccaggtga

SEQ ID NO: 25 - DNA, 51 bp, homo sapiens
taagtgctgtgtggaataaactcgcatagccaatgaaggaagatgaggaac

SEQ ID NO: 26 - DNA, 51 bp, homo sapiens
taagtgctgtgtggaataaactcgcgtagccaatgaaggaagatgaggaac

SEQ ID NO: 27 - DNA, 51 bp, homo sapiens
  misc_feature (26)..(26): n is a, c, or t
cacagcaaatggccgatcatgtaccntcttgcttcatccacccccaaactg

SEQ ID NO: 28 - DNA, 51 bp, homo sapiens
  misc_feature (26)..(26): n is a, c or g
tcatgtttaatatctaactgtatgcntttatgattaaaaaataaagttgaa

SEQ ID NO: 29 - DNA, 52 bp, homo sapiens
  misc_feature (27)..(27): n is a, c, or t
tgaactaggatatttggatataagcantgaactaggatatttggcagaagaa

SEQ ID NO: 30 - DNA, 52 bp, homo sapiens
  misc_feature (27)..(27): n is c, g, or t
accagccagagagatctgcaggaactntactacagcccgtaagaggtgaggc

SEQ ID NO: 31 - DNA, 51 bp, homo sapiens
  misc_feature (26)..(26): n is a, c, or t
tggttaagccagttgttcatacttcntttacaaatataaagatagctgttt

SEQ ID NO: 32 - DNA, 51 bp, homo sapiens
  misc_feature (26)..(26): n is a, g, or t
ttatcataattctacataggattagnggagagaaggatctcaaataaatta

SEQ ID NO: 33 - DNA, 51 bp, homo sapiens
  misc_feature (26)..(26): n is a, c, or g
tctaggtcatgacctggagtcccaangatgaggcagttacccaggtgcctc

SEQ ID NO: 34 - DNA, 51 bp, homo sapiens
  misc_feature (26)..(26): n is a, g, or t
gcaggcaaataagtaaataaacaaangaaatcattatagacagcactgggt

SEQ ID NO: 35 - DNA, 51 bp, homo sapiens
  misc_feature (26)..(26): n is a, g, or t
atgggtcagaacagagtgagtcacanaaaccaccatgcagttcccaggtga

SEQ ID NO: 36 - DNA, 51 bp, homo sapiens
  misc_feature (26)..(26): n is c, g, or t
taagtgctgtgtggaataaactcgcntagccaatgaaggaagatgaggaac

SEQ ID NO: 37 - DNA, 51 bp, homo sapiens
  misc_feature (26)..(26): n is c, g, or t
cagcactttggttcaccttttcttcnatattgtgattctttttgtcataag

SEQ ID NO: 38 - DNA, 51 bp, homo sapiens
  misc_feature (26)..(26): n is a, c, or t
tttcatttgtgctttttctgttcaantttcttgtatgtgtttcatataaca

SEQ ID NO: 39 - DNA, 33 bp, homo sapiens
  misc_feature (17)..(17): n is c, g, or t
agctaggtgtaaagtcngacaagcctgggattg

(19) United States
(12) Patent Application Publication
(10) Pub. No.: US 2012/0065075 A1
(43) Pub. Date: Mar. 15, 2012

(54) METHOD OF DETERMINING A PREDISPOSITION TO ATRIAL FIBRILLATION (AF) IN A SUBJECT

(75) Inventors: Pavel Hamet, Ville Mont-Royal (CA); Johanne Tremblay, Ville Mont-Royal (CA); Jacques De Champlain, Outremont (CA); François de Champlain, (US); Josée Beaudet, (US); Bernard de Champlain, (US); Réginald Nadeau, Montreal (CA); Pierre Larochelle, Brossard (CA); John Chalmers, Longueville (AU); Stephen Macmahon, Pymont (AU)

(73) Assignee: PROGNOMIX INC.

(21) Appl. No.: 13/176,623

(22) Filed: Jul. 5, 2011

Related U.S. Application Data
(60) Provisional application No. 61/360,987, filed on Jul. 2, 2010.

(51) Int. Cl. C40B 20/00; C12Q 1/68; G01N 33/53

(52) U.S. Cl. 506/2; 436/501; 435/6.11

(57) ABSTRACT

The present invention concerns a method of determining a predisposition to atrial fibrillation (AF) in a subject comprising: determining the presence of at least one copy of a risk allele from at least one polymorphic marker in a sample from the subject, wherein the presence of at least one copy of the risk allele is indicative of a predisposition to AF, and wherein said at least one polymorphic marker is: a) rs4674485; b) rs1466560; c) rs1880039; d) rs3849387; e) rs7039; f) rs2952860; g) rs9312515; h) rs1897527; i) rs2299277; j) rs2418828; k) rs2385833; l) rs6717960; m) rs10510266; or n) a substitute polymorphic marker in linkage disequilibrium with any one of the polymorphic markers of a) to m). Also described are kits for determining a predisposition to atrial fibrillation (AF).

### CROSS REFERENCE TO RELATED APPLICATIONS

This application claims benefit of U.S. provisional application Ser. No. 61/360,987, filed on Jul. 2, 2010. The document above is incorporated herein in its entirety by reference.

### STATEMENT REGARDING FEDERALLY SPONSORED RESEARCH OR DEVELOPMENT

N.A.

### FIELD OF THE INVENTION

The present invention relates to the identification of polymorphisms and clinical parameters associated with an increased risk of suffering from atrial fibrillation (AF) and prognostic methods derived therefrom. The present invention also concerns a method for identifying compounds for the prevention of AF.

### REFERENCE TO SEQUENCE LISTING

Pursuant to 37 C.F.R. 1.821(c), a sequence listing is submitted herewith as an ASCII compliant text file named 87581-01_ST25, created on Jul. 5, 2011 and having a size of 6.99 Kb kilobytes. The content of the aforementioned file is hereby incorporated by reference in its entirety.

### BACKGROUND OF THE INVENTION

### Introduction

AF is the most common clinically significant arrhythmia, with a current prevalence in the US of 2.3 million cases and an estimated increase in prevalence so as to reach 15.9 million by the year 2050.¹ AF is associated with a 5-fold increased risk for stroke,² and independent of coexisting diseases, the presence of AF increases the risk for all-cause mortality.³ For instance, in the Action in Diabetes and Vascular Disease (ADVANCE) study⁴, AF was associated with a 61% greater risk of all-cause mortality and comparable higher risks of cardiovascular death, stroke, and heart failure.⁴

There is strong evidence that indicates that genetic factors play a role in the development of AF (reviewed in ^{5;6}). For instance, in the Framingham cohort, history of AF in at least 1 parent increased the risk of AF in the offspring by almost two-fold.⁷ Moreover, several monogenic forms of the disease bearing mutations in KCNQ1⁸ and KCNE2⁹ genes have been described, and linkage studies have also identified several loci in families with AF.^{10;11} At a population level, it has been found that up to 80% of individuals with AF are related to some degree to another individual with AF.¹² In addition, recent genome wide association studies (GWAS)^{13;14} have identified single nucleotide polymorphisms (SNPs) associated with higher susceptibility to develop AF. The prevalence of these genetic variants^{13;14} in the HapMapm™ population with ancestry from northern and western Europe (HapMap-CEU) ranges from 0.11 to 0.38,¹⁵ and the associated risk for carriers may increase up to 2.3 times.¹³

In contrast to monogenic disorders, which may be a rare cause of the disease in the general population, genetic risk variants that confer predisposition/susceptibility to common forms of AF (e.g., SNPs) may be highly prevalent in the general population. Genetically predisposed individuals may then develop the disease when exposed to certain, triggering environmental factors.

Hypertension is the most common condition associated with AF, being present in more than 50% of cases.¹⁶ AF occurs more often in hypertensive subjects with associated cardiac structural abnormalities such as left ventricular hypertrophy. However, not all hypertensive individuals, including those with similar blood pressure or alterations in cardiac structure develop this arrhythmia.^{6;17} Currently, it is uncertain if there are genetic variants that may increase the risk of AF in hypertensive individuals and whether these genetic factors interact with other clinical or metabolic risk factors for AF. Thus, a multivariate approach including neuroendocrine and genetic markers would add meaningful information to traditional risk factors.

Due to the nature of paroxysmal AF (generally defined by recurrent episodes that self terminate in less than 7-days) it can be very difficult to diagnose. When the patient seeks medical attention due to disease-related symptoms, such as palpitations, chest pain, shortness of breath, dizziness, heart failure, transient ischemic attacks or even stroke, normal heart rhythm may already be restored precluding diagnosis of the arrhythmia. In these cases cardiac rhythm monitoring is frequently applied in the attempt to diagnose the condition. The cardiac rhythm is commonly monitored continuously for 24 to 48 hours. Unfortunately AF episodes are unpredictable and frequently missed by this approach. The opportunity to diagnose the arrhythmia, institute recommended therapy, and possibly prevent a debilitating first or recurrent stroke may be missed with devastating results to the patient. Prolonged and more complex cardiac rhythm monitoring measures are available and applied occasionally when the suspicion of AF is very strong. These tests are expensive, the diagnostic yield with current approach is often low, and they are used sparingly for this indication.

Thus, there remains a need for an understanding of the factors leading to increased predisposition to AF and/or stroke.

The present description refers to a number of documents, the content of which is herein incorporated by reference in their entirety.

### SUMMARY OF THE INVENTION

The present invention relates to the discovery that certain polymorphic markers are associated with AF and indirectly with a risk of stroke. In addition, the present invention relates to the discovery that these polymorphic markers, when associated with particular clinical and neuroendocrine factors, increase the specificity of the diagnostic method and therefore, the likelihood to identify subjects at higher risk of developing AF.

The identification of genetic markers as well as other factors (e.g., clinical factors and neuroendocrine factors) is useful in identifying which subjects are at particularly high risk for AF and thus subsequent stroke. In addition, prophylactic/preventive treatment can be administered to subjects which are most at risk of AF. Finally, the identification of genetic factors associated with AF can lead to the identification of new targets for prevention and treatment of AF, as well as the development of novel therapeutic/prophylactic measures.

Accordingly, the present invention relates to a method of determining a predisposition to atrial fibrillation (AF) in a subject comprising: determining the presence of at least one copy of a risk allele from at least one polymorphic marker in a sample from the subject, wherein the presence of at least one copy of the risk allele is indicative of a predisposition to AF, and wherein said at least one polymorphic marker is: a) rs4674485; b) rs1466560; c) rs1880039; d) rs3849387; e) rs7039; f) rs2952860; g) rs9312515; h) rs1897527; i) rs2299277; j) rs2418828; k) rs2385833; l) rs6717960; m) rs10510266; or n) a substitute polymorphic marker in linkage disequilibrium with any one of the polymorphic markers of a) to m).

The present invention also describes a method of determining a susceptibility or predisposition to atrial fibrillation (AF) in a subject comprising: comparing at least one polymorphic marker in a sample from the subject to a reference sequence or control sample, wherein a difference between at least one copy of an allele of the polymorphic marker from the subject as compared to the reference sequence or control sample is indicative of a susceptibility to AF and wherein said at least one polymorphic marker is: a) rs4674485; b) rs1466560; c) rs1880039; d) rs3849387; e) rs7039; f) rs2952860; g) rs9312515; h) rs1897527; i) rs2299277; j) rs2418828; k) rs2385833; l) rs6717960; m) rs10510266; and n) a substitute polymorphic marker in linkage disequilibrium with any one of the polymorphic markers of a) to m).

In an embodiment, the method of the present invention comprises comparing a sequence of at least one polymorphic marker from the subject to a reference sequence not associated with AF. In an embodiment, the above-mentioned reference sequence or control sequence is from an hypertensive subject (or subjects) not susceptible to AF (i.e. as identified by other known predictors (e.g., clinical and/or neuroendocrine predictors such as a higher left atrial volume, and/or a higher pulse pressure and/or a lower ANP level, etc.). In an embodiment, the reference sequence or control sequence is from a normotensive subject (or subjects) not susceptible to AF.

In an embodiment of the method of the present invention, the sample is a nucleic acid sample. In another embodiment, the sample is a protein sample.

In an embodiment of the method of the present invention, the reference sequence is a nucleic acid sequence comprising the sequence of: a) SEQ ID NO:2 for the polymorphic marker rs4674485; b) SEQ ID NO:4 for the polymorphic marker rs1466560; c) SEQ ID NO:14 for the polymorphic marker rs1880039; d) SEQ ID NO:15 for the polymorphic marker rs3849387; e) SEQ ID NO:10 for the polymorphic marker rs7039; f) SEQ ID NO:17 for the polymorphic marker rs2952860; g) SEQ ID NO:19 for the polymorphic marker rs9312515; h) SEQ ID NO:22 for the polymorphic marker rs1897527; i) SEQ ID NO:23 for the polymorphic marker rs2299277; j) SEQ ID NO:25 for the polymorphic marker rs2418828; k) SEQ ID NO:6 for the polymorphic marker rs2385833; l) SEQ ID NO:7 for the polymorphic marker rs6717960; m) SEQ ID NO:11 for the polymorphic marker rs10510266; or n) a complement of any one of a) to m).

In an embodiment of method of the present invention, the risk allele comprises the nucleic acid sequence of: a) SEQ ID NO:27 for the polymorphic marker rs4674485; b) SEQ ID NO:28 for the polymorphic marker rs1466560; c) SEQ ID NO:33 for the polymorphic marker rs1880039; d) SEQ ID NO:34 for the polymorphic marker rs3849387; e) SEQ ID NO:31 for the polymorphic marker rs7039; f) SEQ ID NO:32 for the polymorphic marker rs2952860; g) SEQ ID NO:37 for the polymorphic marker rs9312515; h) SEQ ID NO:38 for the polymorphic marker rs1897527; i) SEQ ID NO:35 for the polymorphic marker rs2299277; j) SEQ ID NO:36 for the polymorphic marker rs2418828; k) SEQ ID NO:29 for the polymorphic marker rs2385833; l) SEQ ID NO:30 for the polymorphic marker rs6717960; m) SEQ ID NO:39 for the polymorphic marker rs10510266; or n) a complement of any one of a) to m).

In a related aspect, the risk allele of the present invention comprises the nucleic acid sequence of: a) SEQ ID NO:1 for the polymorphic marker rs4674485; b) SEQ ID NO:3 for the polymorphic marker rs1466560; c) SEQ ID NO:13 for the polymorphic marker rs1880039; d) SEQ ID NO:16 for the polymorphic marker rs3849387; e) SEQ ID NO:9 for the polymorphic marker rs7039; f) SEQ ID NO:18 for the polymorphic marker rs2952860; g) SEQ ID NO:20 for the polymorphic marker rs9312515; h) SEQ ID NO:21 for the polymorphic marker rs1897527; i) SEQ ID NO:24 for the polymorphic marker rs2299277; j) SEQ ID NO:26 for the polymorphic marker rs2418828; k) SEQ ID NO:5 for the polymorphic marker rs2385833; l) SEQ ID NO:8 for the polymorphic marker rs6717960; m) SEQ ID NO:12 for the polymorphic marker rs10510266; or n) a complement of any one of a) to m).

In an embodiment, the method comprises determining the presence of a reference allele or reference sequence in a sample by determining the presence of at least one polypeptide or protein encoded by a nucleic acid sequence comprising the nucleic acid sequence of: a) SEQ ID NO:2 for the polymorphic marker rs4674485; b) SEQ ID NO:4 for the polymorphic marker rs1466560; c) SEQ ID NO:14 for the polymorphic marker rs1880039; d) SEQ ID NO:15 for the polymorphic marker rs3849387; e) SEQ ID NO:10 for the polymorphic marker rs7039; f) SEQ ID NO:17 for the polymorphic marker rs2952860; g) SEQ ID NO:19 for the polymorphic marker rs9312515; h) SEQ ID NO:22 for the polymorphic marker rs1897527; i) SEQ ID NO:23 for the polymorphic marker rs2299277; j) SEQ ID NO:25 for the polymorphic marker rs2418828; k) SEQ ID NO:6 for the polymorphic marker rs2385833; l) SEQ ID NO:7 for the polymorphic marker rs6717960; m) SEQ ID NO:11 for the polymorphic marker rs10510266; or n) a complement of any one of a) to m).

In an embodiment, the method comprises determining the presence of the risk allele in the sample by determining the presence of at least one polypeptide or protein associated with a predisposition to AF encoded by a nucleic acid sequence comprising the nucleic acid sequence of: a) SEQ ID NO:27 for the polymorphic marker rs4674485; b) SEQ ID NO:28 for the polymorphic marker rs1466560; c) SEQ ID NO:33 for the polymorphic marker rs1880039; d) SEQ ID NO:34 for the polymorphic marker rs3849387; e) SEQ ID NO:31 for the polymorphic marker rs7039; f) SEQ ID NO:32 for the polymorphic marker rs2952860; g) SEQ ID NO:37 for the polymorphic marker rs9312515; h) SEQ ID NO:38 for the polymorphic marker rs1897527; i) SEQ ID NO:35 for the polymorphic marker rs2299277; j) SEQ ID NO:36 for the polymorphic marker rs2418828; k) SEQ ID NO:29 for the polymorphic marker rs2385833; l) SEQ ID NO:30 for the polymorphic marker rs6717960; m) SEQ ID NO:39 for the polymorphic marker rs10510266; or n) a complement of any one of a) to m).

In an embodiment, the method comprises determining the presence of the risk allele in the sample by determining the presence of at least one polypeptide or protein associated with a predisposition to AF encoded by a nucleic acid sequence comprising the nucleic acid sequence of: a) SEQ ID NO:1 for the polymorphic marker rs4674485; b) SEQ ID NO:3 for the polymorphic marker rs1466560; c) SEQ ID NO:13 for the polymorphic marker rs1880039; d) SEQ ID NO:16 for the polymorphic marker rs3849387; e) SEQ ID NO:9 for the polymorphic marker rs7039; f) SEQ ID NO:18 for the polymorphic marker rs2952860; g) SEQ ID NO:20 for the polymorphic marker rs9312515; h) SEQ ID NO:21 for the polymorphic marker rs1897527; i) SEQ ID NO:24 for the polymorphic marker rs2299277; j) SEQ ID NO:26 for the polymorphic marker rs2418828; k) SEQ ID NO:5 for the polymorphic marker rs2385833; l) SEQ ID NO:8 for the polymorphic marker rs6717960; m) SEQ ID NO:12 for the polymorphic marker rs10510266; or n) a complement of any one of a) to m).

In an embodiment of the method of the present invention, the at least one polymorphic marker comprises rs4674485. In an embodiment, the at least one polymorphic marker comprises rs1466560. In another embodiment, the at least one polymorphic marker is at least one substitute polymorphic marker in linkage disequilibrium with any one of the polymorphic markers of a) to j).

In an embodiment of the present invention the at least one substitute polymorphic marker is rs2385833 or rs6717960, wherein rs2385833 or rs6717960 are in linkage disequilibrium with rs4674485. In a embodiment, the at least one substitute polymorphic marker is rs10510266, wherein rs10510266 is in linkage disequilibrium with rs1466560.

In another aspect, the method of the present invention further comprises determining the presence of at least one of: (a) pulse pressure; (b) left atrial volume; and (c) atrial natriuretic peptide (ANP) concentration, wherein a higher pulse pressure and/or left atrial volume as compared to normotensive subjects or a decrease in ANP concentration as compared to hypertensive individuals is indicative of an increased risk of suffering from atrial fibrillation.

In a related aspect, the present invention concerns a method of determining a predisposition to atrial fibrillation (AF) in a subject suffering from hypertension comprising determining at least one of: (a) pulse pressure; (b) left atrial volume; and (c) atrial natriuretic peptide (ANP) concentration, in said subject, wherein a higher pulse pressure and/or higher left atrial volume and/or a lower concentration of ANP as compared to an hypertensive subject not suffering from AF is indicative of an increased risk of suffering from AF. In a specific embodiment, the method determines at least left atrial volume and at least one of pulse pressure; and atrial natriuretic peptide.

In an embodiment of the present invention, the decrease in ANP concentration is equivalent to the ANP concentration in normotensive individuals.

The determination of pulse pressure, left atrial volume and/or ANP concentration can be used alone or in combination with the determination of the presence of one or more of the above-mentioned polymorphic markers. The determination of the presence of polymorphic markers and of one or more of pulse pressure, left atrial volume and ANP concentration is advantageous as it increases the specificity of the method.

In an embodiment of the method of the present invention at least two of pulse pressure, left atrial volume and ANP blood concentration are determined. In another embodiment, pulse pressure, left atrial volume and ANP blood concentration (e.g., whole blood, plasma, serum) are determined.

In a particular embodiment of the method of the present invention, AF is paroxysmal AF. In an embodiment, the subject suffers from hypertension.

In one aspect, the method of the present invention has a specificity of at least 80%. In an embodiment, the method has a specificity of at least 85%.

In a related aspect, the method of the present invention further comprises determining whether the subject is in need of preventive medication.

In an embodiment, the method further comprises assessing the subject's probability of response to a therapeutic agent for preventing and/or ameliorating symptoms associated with AF and/or stroke.

In a further aspect, the present invention concerns a kit for practicing the method of the present invention.

In an embodiment, the present invention relates to a kit for determining a predisposition to atrial fibrillation (AF) in a subject comprising: reagents for determining the presence of at least one copy of a risk allele from at least one polymorphic marker in a sample from the subject (e.g., one or more probes and/or primers for determining the presence of one or more of the above-mentioned polymorphic markers (risk allele or reference allele), wherein the presence of at least one copy of the risk allele is indicative of a predisposition to AF and wherein the at least one polymorphic marker is: a) rs4674485; b) rs1466560; c) rs1880039; d) rs3849387; e) rs7039; f) rs2952860; g) rs9312515; h) rs1897527; i) rs2299277; j) rs2418828; k) rs2385833; l) rs6717960; m) rs10510266; or n) a substitute polymorphic marker in linkage disequilibrium with any one of the polymorphic markers of a) to m). In an embodiment, the kit further comprises instructions for determining a predisposition to AF.

In an embodiment, the kit may comprise one or more primers or probes for determining the presence of one or more of the above-mentioned polymorphic markers (risk allele or reference allele) as well as buffers or reagents for the detection of amplification or hybridization products depending on the selected method.

In an embodiment, the kit of the present invention comprises reagents for determining the presence of a reference sequence of a polymorphic maker of the present invention, wherein said reference sequence is not associated with AF.

In an embodiment, the above-mentioned reference sequence or control sequence is from an hypertensive subject (or subjects) not susceptible to AF (i.e. as identified by other known predictors (e.g., clinical and/or neuroendocrine predictors such as a higher left atrial volume, and/or a higher pulse pressure and/or a lower ANP level, etc.). In an embodiment, the reference sequence or control sequence is from a normotensive subject (or subjects) not susceptible to AF.

In an embodiment of the kit of the present invention, the sample is a nucleic acid sample. In another embodiment, the sample is a protein sample.

In an embodiment of the kit of the present invention, the reference sequence is a nucleic acid sequence comprising the sequence of: a) SEQ ID NO:2 for the polymorphic marker rs4674485; b) SEQ ID NO:4 for the polymorphic marker rs1466560; c) SEQ ID NO:14 for the polymorphic marker rs1880039; d) SEQ ID NO:15 for the polymorphic marker rs3849387; e) SEQ ID NO:10 for the polymorphic marker rs7039; f) SEQ ID NO:17 for the polymorphic marker rs2952860; g) SEQ ID NO:19 for the polymorphic marker rs9312515; h) SEQ ID NO:22 for the polymorphic marker rs1897527; i) SEQ ID NO:23 for the polymorphic marker rs2299277; j) SEQ ID NO:25 for the polymorphic marker rs2418828; k) SEQ ID NO:6 for the polymorphic marker rs2385833; l) SEQ ID NO:7 for the polymorphic marker rs6717960; m) SEQ ID NO:11 for the polymorphic marker rs10510266; or n) a complement of any one of a) to m).

In an embodiment of the kit of the present invention, the risk allele comprises the nucleic acid sequence of: a) SEQ ID NO:27 for the polymorphic marker rs4674485; b) SEQ ID NO:28 for the polymorphic marker rs1466560; c) SEQ ID NO:33 for the polymorphic marker rs1880039; d) SEQ ID NO:34 for the polymorphic marker rs3849387; e) SEQ ID NO:31 for the polymorphic marker rs7039; f) SEQ ID NO:32 for the polymorphic marker rs2952860; g) SEQ ID NO:37 for the polymorphic marker rs9312515; h) SEQ ID NO:38 for the polymorphic marker rs1897527; i) SEQ ID NO:35 for the polymorphic marker rs2299277; j) SEQ ID NO:36 for the polymorphic marker rs2418828; k) SEQ ID NO:29 for the polymorphic marker rs2385833; l) SEQ ID NO:30 for the polymorphic marker rs6717960; m) SEQ ID NO:39 for the polymorphic marker rs10510266; or n) a complement of any one of a) to m).

In an embodiment, the risk allele comprises the nucleic acid sequence of: a) SEQ ID NO:1 for the polymorphic marker rs4674485; b) SEQ ID NO:3 for the polymorphic marker rs1466560; c) SEQ ID NO:13 for the polymorphic marker rs1880039; d) SEQ ID NO:16 for the polymorphic marker rs3849387; e) SEQ ID NO:9 for the polymorphic marker rs7039; f) SEQ ID NO:18 for the polymorphic marker rs2952860; g) SEQ ID NO:20 for the polymorphic marker rs9312515; h) SEQ ID NO:21 for the polymorphic marker rs1897527; i) SEQ ID NO:24 for the polymorphic marker rs2299277; j) SEQ ID NO:26 for the polymorphic marker rs2418828; k) SEQ ID NO:5 for the polymorphic marker rs2385833; l) SEQ ID NO:8 for the polymorphic marker rs6717960; m) SEQ ID NO:12 for the polymorphic marker rs10510266; or n) a complement of any one of a) to m).

In an embodiment, the kit comprises reagents (e.g., an antibody) for determining the presence of at least one polypeptide or protein, wherein the polypeptide or protein is encoded by a nucleic acid sequence comprising the reference sequence: a) SEQ ID NO:2 for the polymorphic marker rs4674485; b) SEQ ID NO:4 for the polymorphic marker rs1466560; c) SEQ ID NO:14 for the polymorphic marker rs1880039; d) SEQ ID NO:15 for the polymorphic marker rs3849387; e) SEQ ID NO:10 for the polymorphic marker rs7039; f) SEQ ID NO:17 for the polymorphic marker rs2952860; g) SEQ ID NO:19 for the polymorphic marker rs9312515; h) SEQ ID NO:22 for the polymorphic marker rs1897527; i) SEQ ID NO:23 for the polymorphic marker rs2299277; j) SEQ ID NO:25 for the polymorphic marker rs2418828; k) SEQ ID NO:6 for the polymorphic marker rs2385833; l) SEQ ID NO:7 for the polymorphic marker rs6717960; m) SEQ ID NO:11 for the polymorphic marker rs10510266; or n) a complement of any one of a) to m).

In another embodiment, the kit comprises reagents (e.g., an antibody) for determining the presence of at least one polypeptide or protein is encoded by associated with a predisposition to AF, wherein the polypeptide or protein is encoded by a nucleic acid sequence comprising the nucleic acid sequence of: a) SEQ ID NO:27 for the polymorphic marker rs4674485; b) SEQ ID NO:28 for the polymorphic marker rs1466560; c) SEQ ID NO:33 for the polymorphic marker rs1880039; d) SEQ ID NO:34 for the polymorphic marker rs3849387; e) SEQ ID NO:31 for the polymorphic marker rs7039; f) SEQ ID NO:32 for the polymorphic marker rs2952860; g) SEQ ID NO:37 for the polymorphic marker rs9312515; h) SEQ ID NO:38 for the polymorphic marker rs1897527; i) SEQ ID NO:35 for the polymorphic marker rs2299277; j) SEQ ID NO:36 for the polymorphic marker rs2418828; k) SEQ ID NO:29 for the polymorphic marker rs2385833; l) SEQ ID NO:30 for the polymorphic marker rs6717960; m) SEQ ID NO:39 for the polymorphic marker rs10510266; or n) a complement of any one of a) to m).

In another embodiment, the kit comprises reagents (e.g., an antibody) for determining the presence of at least one polypeptide or protein associated with a predisposition to AF, wherein the polypeptide or protein is encoded by a nucleic acid sequence comprising the nucleic acid sequence of: a) SEQ ID NO:1 for the polymorphic marker rs4674485; b) SEQ ID NO:3 for the polymorphic marker rs1466560; c) SEQ ID NO:13 for the polymorphic marker rs1880039; d) SEQ ID NO:16 for the polymorphic marker rs3849387; e) SEQ ID NO:9 for the polymorphic marker rs7039; f) SEQ ID NO:18 for the polymorphic marker rs2952860; g) SEQ ID NO:20 for the polymorphic marker rs9312515; h) SEQ ID NO:21 for the polymorphic marker rs1897527; i) SEQ ID NO:24 for the polymorphic marker rs2299277; j) SEQ ID NO:26 for the polymorphic marker rs2418828; k) SEQ ID NO:5 for the polymorphic marker rs2385833; l) SEQ ID NO:8 for the polymorphic marker rs6717960; m) SEQ ID NO:12 for the polymorphic marker rs10510266; or n) a complement of any one of a) to m).

In an embodiment the kit of the present invention comprises reagents for determining the presence of the polymorphic marker rs4674485. In an embodiment, the kit comprises reagents for determining the presence of rs1466560. In another embodiment, the kit comprises reagents for determining the presence of at least one substitute polymorphic marker in linkage disequilibrium with any one of the above-mentioned polymorphic markers of a) to j).

In an embodiment, the at least one substitute polymorphic marker is rs2385833 or rs6717960, wherein rs2385833 or rs6717960 are in linkage disequilibrium with rs4674485.

In an embodiment, the at least one substitute polymorphic marker is rs10510266, wherein rs10510266 is in linkage disequilibrium with rs1466560.

In another aspect, the kit of the present invention further comprises reagents and/or instructions for determining the presence of at least one of: (a) pulse pressure; (b) left atrial volume; and (c) atrial natriuretic peptide (ANP) concentration in said subject.

In a related aspect, the present invention concerns a kit for determining a predisposition to atrial fibrillation (AF) in a subject suffering from hypertension comprising reagents and/or instructions for determining at least one of: (a) pulse pressure; (b) left atrial volume; and (c) atrial natriuretic peptide (ANP) concentration, in said subject. In a specific embodiment, the kit comprises reagents and/or instructions for determining left atrial volume and at least one of pulse pressure; and atrial natriuretic peptide.

Thus, in accordance with the present invention, the kit may comprises reagents and/or instructions for the determination of pulse pressure, left atrial volume and/or ANP concentration alone or in combination with reagents for the determination of the presence of one or more of the above-mentioned polymorphic markers.

In an embodiment, the kit of the present invention comprises reagents and/or instructions for determining at least two of pulse pressure, left atrial volume and ANP blood concentration (e.g., whole blood, plasma, serum). In another embodiment, the kit comprises reagents and/or instructions for determining pulse pressure, left atrial volume and ANP blood concentration.

In a particular embodiment, the kit of the present invention is for determining a predisposition to paroxysmal AF. In an embodiment, the kit is for determining a predisposition to AF in subject suffering from hypertension.

Reagents of the present invention include, but are not limited to, oligonucleotide primers and probes for determining the presence of at least one copy of a risk allele or reference sequence of at least one polymorphic marker of the present invention. Alternatively, antibodies, ligands and substrates may be used to detect polypeptides and proteins encoded by a nucleic acid sequence comprising the polymorphic marker of the present invention.

Thus, in an embodiment, the kit of the present invention includes at least one oligonucleotide (e.g., primer or probe) for determining the presence of at least one copy of a risk allele of at least one polymorphic marker of the present invention. In an embodiment, the kit includes at least one oligonucleotide for determining the presence of at least one copy of a reference allele of the polymorphic marker of the present invention.

The present invention further relates to the use of the above-mentioned kit of the present invention, for determining a predisposition to AF in a subject.

In an embodiment of the present invention, the presence of at least one copy of an allele of at least two of the above-mentioned polymorphic markers is determined. In an embodiment, the presence of at least one copy of at least one allele of at least three of the above-mentioned polymorphic markers is determined. In an embodiment, the presence at least one copy of at least one allele of at least four of the above-mentioned polymorphic markers is determined. In an embodiment, the presence of at least one copy of at least one allele of at least five of the above-mentioned polymorphic markers is determined. In an embodiment, the presence of at least one copy of at least one allele of at least six of the above-mentioned polymorphic markers is determined. In an embodiment, the presence of at least one copy of at least one allele of at least seven of the above-mentioned polymorphic markers is determined. In an embodiment, the presence of at least one copy of at least one allele of at least eight of the above-mentioned polymorphic markers is determined. In an embodiment, the presence of at least one copy of at least one allele of at least nine of the above-mentioned polymorphic markers is determined. In an embodiment, the presence of at least one copy of at least one allele of all of the above-mentioned polymorphic markers is determined.

In a further embodiment, the presence of one or more additional polymorphic markers (e.g., KCNQ1, KCNE2) or biomarkers (e.g., clinical factors, neuroendocrine factors, etc.) is determined.

In an embodiment of the above-described methods, a susceptibility to AF is identified by determining the presence in a sample from a subject of at least one allele of at least one substitute polymorphic marker in linkage disequilibrium with a) rs4674485; b) rs1466560; c) rs1880039); d) rs3849387; e) rs7039; f) rs2952860; g) rs9312515; h) rs1897527; i) rs2299277; or j) rs2418828.

In particular embodiments of the invention, linkage disequilibrium (LD) is defined by a specific quantitative cutoff. As described in detail herein, linkage disequilibrium can be quantitatively determined by measures such as r² and |D′|. As a consequence, certain embodiments of the invention relate to substitute markers in linkage disequilibrium by a measure within a certain range specified by particular values of r² and/or |D′|. In an embodiment, LD is characterized by numerical values for r² of greater than 0.1. In another embodiment, LD is characterized by numerical values for r² of greater than 0.5. In another embodiment, LD is characterized by numerical values for r² of greater than 0.8. In another embodiment, LD is characterized by numerical values for r² of 0.9 or more.

Other cutoff values for r² are also contemplated, as described in more detail herein.

In certain embodiments, LD is characterized by certain cutoff values for r² and/or |D′|. In one such embodiment, LD is characterized by values for r² and/or |D′| of greater than 0.2 and 0.8, respectively. Other combinations and permutations of these or other measures of LD are possible to practice the invention, and are also contemplated and within scope of the invention.

In a particular embodiment, the above-mentioned methods comprises determining the presence of at least one of rs4674485 and rs1466560 or a substitute polymorphic marker in linkage disequilibrium therewith. In an embodiment, the substitute polymorphic marker in linkage disequilibrium with rs4674485 is rs2385833 (wherein SEQ ID NO:6 is a reference sequence for this marker and wherein SEQ ID NOs: 5 and 29 are risk variants for this marker) or rs6717960 (wherein SEQ ID NO:7 is a reference sequence for this marker and wherein SEQ ID NOs: 8 and 30 are risk variants for this marker). In an embodiment, the substitute polymorphic marker in linkage disequilibrium with rs1466560 is rs10510266 (wherein SEQ ID NO:11 is a reference sequence for this marker and wherein SEQ ID NOs: 12 and 39 are risk variants for this marker).

The determination of the presence of an allele associated with AF in a sample from a subject in accordance with the present invention can be done using any suitable methods. One non-limiting example is genotyping. Genotyping can be performed by a variety of methods including allele-specific probe hybridization, allele-specific primer extension, allele-specific amplification, nucleic acid sequencing, 5′-exonuclease digestion, molecular beacon assay, oligonucleotide ligation assay, size analysis, and single-stranded conformation analysis (SSCA).

Furthermore a variety of detection means may be used, including, but not limited to, one or a combination of the following: radiolabelling (e.g., ³²P, ¹⁴C, ³H), antibody-based detection, fluorescence, chemiluminescence, spectroscopic methods (e.g., generation of a product with altered spectroscopic properties), various reporter enzymes or proteins (e.g., horseradish peroxidase, green fluorescent protein), specific binding reagents (e.g., biotin/(strept)avidin), and others.

Other examples of suitable methods used to detect or quantify altered gene, RNA or protein expression or sequence include: amplification and/or binding to specific ligands (such as antibodies), Southern blot (for DNAs), Northern blot (for RNAs), single-stranded conformation analysis (SSCA), pulse field gel electrophoresis (PFGE), fluorescent in situ hybridization (FISH), gel migration, clamped denaturing gel electrophoresis (CDGE), heteroduplex analysis, RNase protection, chemical mismatch cleavage, ELISA, radio-immunoassays (RIA) and immuno-enzymatic assays (IEMA).

Some of these approaches (e.g., SSCA and CDGE) are based on a change in electrophoretic mobility of the nucleic acids, as a result of the presence of an altered sequence. According to these techniques, the altered sequence is visualized by a shift in mobility on gels. The fragments may then be sequenced to confirm the alteration. Some others are based on specific hybridization between nucleic acids from the subject and a probe specific for wild-type or altered gene or RNA. The probe may be in suspension or immobilized on a substrate. The probe is typically labeled to facilitate detection of hybrids. By “specific hybridization” is intended a hybridization under stringent conditions which allows to detect distinctively the desired allele (risk allele/sequence or reference allele/sequence) as opposed to a non-specific hybridization which cannot distinguish the risk allele (sequence) from the reference allele (sequence).

Some of these approaches are particularly suited for assessing a polypeptide sequence or its expression level, such as Western blot, immunoblot, enzyme-linked immunosorbant assay (ELISA), radioimmunoassay (RIA), immunoprecipitation, surface plasmon resonance, chemiluminescence, fluorescent polarization, phosphorescence, immunohistochemical analysis, matrix-assisted laser desorption/ionization time-of-flight (MALDI-TOF) mass spectrometry, microcytometry, microarray, microscopy, fluorescence activated cell sorting (FACS), flow cytometry, and assays based on a property of the protein including, but not limited to, DNA binding, ligand binding, or interaction with other protein partners. The latter requires the use of a ligand specific for the polypeptide, more preferably of a specific antibody.

Amplification may be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Preferred techniques use allele-specific PCR or PCR-SSCP. Amplification usually requires the use of specific nucleic acid primers, to initiate the reaction.

As indicated above, the present invention describes methods whereby the detection of particular alleles of particular polymorphic markers, combined or not with the assessment of certain clinical and/or neuroendocrine markers is indicative of a susceptibility to AF and/or indirectly to stroke. Such prognostic or predictive assays can also be used to determine a prophylactic treatment for AF or stroke prior to the onset of symptoms associated therewith.

Accordingly, in an embodiment, the above-mentioned method further comprises classifying the test subject based on its relative risk of suffering from AF. In an embodiment, the above-mentioned method further comprises selecting an appropriate prophylactic treatment or drug for preventing AF. In another embodiment, the above-mentioned method further comprises selecting an appropriate prophylactic treatment or drug for preventing a stroke based on the susceptibility (relative risk) of suffering from AF.

In an embodiment, the particular number of polymorphic markers of the present invention to be detected, their specific combination and the number of clinical and/or neuroendocrine factors to be used in the above-described method are selected to obtain a specificity which compensate for the relative risk of not being administered a proper prophylactic treatment to prevent stroke and/or AF. In general, the prophylactic treatment (e.g., anticoagulants such as daiteparin (e.g., Fragmin™), danaparoid (e.g., Orgaran™), enoxaparin (e.g., Lovenox™), heparin (various), tinzaparin (e.g., Innohep™) and warfarin (e.g., Coumadin™)) selected once a particular risk of suffering from AF or stroke is determined should have a lower relative risk (probability of occurrence of side effect and/or gravity of side effects) than the risk of suffering from AF or stroke. (i.e. risk of suffering from AF or stroke should outweigh the risk (e.g., hemorrhaging) associated with prophylactic treatment).

Thus in an embodiment, the specificity of the above-mentioned method is at least 70%, at least 75%, at least 80%, at least 85%, at least 90%, at least 9.5% or at least 98%. In a particular embodiment, the specificity of the above-mentioned method is at least 80%.

The methods of the present invention may further comprise a step of administering to a subject identified as being at increased risk of suffering from AF (or stroke) a composition comprising at least one therapeutic or prophylactic agent effective to treat or prevent AF (or stroke), or prevent symptoms associated with AF (or stroke). Thus, the present invention can be used to determine whether an individual is suitable for a particular treatment regimen.

In one embodiment, the therapeutic agent is an anticoagulant, an anti-arrhythmic agent, or a heart rhythm control agent.

In another embodiment, the therapeutic agent is at least one of warfarin, heparin, low molecular weight heparins, vitamin K antagonists, factor Xa inhibitors, factor Va inhibitors, factor Villa inhibitors, thrombin inhibitors, class I to V antiarrhythmic agents, sodium channel blockers, beta blockers, potassium channel blockers, calcium channel blockers, IKr blockers, IKs blockers, Ito blockers, Ikatp blockers, RyR2 modifiers, 5-HT4 antagonists, IKAch blockers, multiple channel blockers, Ina blockers, ICal blockers, Ikur blockers, gap junction modifiers, ximelagatran, aspirin, enoxaparin, dalteparin, tinzaparin, ardeparin, nadroparin, reviparin, fondaparinux, idraparinux, lepirudin, bivalirudin, argatroban, lepirudin, dabigatran, ximelagatran, Tifacogin, drotrecogin, razaxaban, apixaban, otamixaban, rivaroxaban, betrixaban, LY517717, YM150, DU-176b, danaparoid, disopyramide, moricizine, procainamide, quinidine, lidocaine, phenytoin, encainide, mexiletine, tocainide, flecainide, propafenone, ajmaline, cibenzoline, detajmium, esmolol, propranolol, metoprolol, alprenolol, atenolol, carvedilol, bisoprolol, penbutolol, acebutolol, nadolol, pindololol, labetalol, oxprenotol, timolol, betaxolol, cartelol, sotalol, levobunolol, dofetilide, E-4031, amiodarone, azimilide, bretylium, tedisamil, ibutilide, sematilide, N-acetyl procainamide, nifekalant hydrochloride, vemakalant, Almokalant, clamikalant, dronedarone, celivarone, ranolazine, GsMtx4, XEND0101/2, PM 101, ATI-2042, AVE0118, AZD7009, BRL-32872, HMR 1556, HMR 1883, L768673, S9947, S20951, NIP-141/142, JTV519, GAP-134, RS-100302, Tecadenoson, Selodenoson, pilsicainide, cariporide, eniporide, sabiporide, zoniporide, pirfenidone, rotigaptide, dantrolene, ambasilide, verapamil, mibefradil, Ersentilide, Trecetilide, diltiazem, digoxin, adenosine, drofetilide, procainamide, amiodarone, profafenone and flecainide.

Other features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows clinical and neuroendocrine predictors of AF among hypertensive individuals after stepwise multivariate logistic regression analysis. FIG. 1 A: Left atrial volume (2-D echocardiogram); B: Pulse pressure; and C: Atrial natriuretic peptide (ANP). FIGS. 1 A to C represent means and 95% confidence intervals (95% CI). Normotensive individuals (NT) are given as a reference. *P-value<0.01 for hypertensive individuals who developed AF (HT+AF) vs. hypertensives without history of AF (HT);

FIG. 2 shows matrix correlation of clinical and neurohormonal predictors of AF. FIG. 2A: matrix correlation between pulse pressure and LA volume. FIG. 2B: matrix correlation between ANP and LA volume. FIG. 2C: matrix correlation between ANP and pulse pressure. (LA volume: Left Atrial volume (ml/m²); pulse pressure (mmHg); ANP: Atrial natriuretic peptide (fmol/ml)) among hypertensive individuals with (square; HT+AF) or without (diamond; HT) history of AF. Normotensive individuals (cross; NT) are provided as a reference. Line represents best linear fit of the data for each respective group (HT+AF: full line; HF: scattered line; NT: dotted line). Pearson correlatiOn coefficient for HT patients (PP and ANP: 0.57; P<0.0001; PP and LA volume: 0.25; P<0.01; LA volume and ANP: 0.36; P<0.001) and for HT+AF (PP and ANP: 0.01; P=0.6; PP and LA volume: 0.29; P<0.01; LA volume and ANP: 0.15; P=0.1);

FIG. 3 shows the genomic context for rs4674485 [A/G] (chromosome 2q35) associated with development of AF among hypertensives. Panel A shows probability of association (plotted as −log 10 p) for rs4674485 (The highest p-value is indicated by vertical dotted lines) and surrounding SNPs. Panel B shows LD pattern from HapMap™-CEU in a region (dashed lines) +/−200 Kb around rs4674485 (Haploview). Panel C shows LD information for SNPs in discovery (rs4674485) and replication cohorts (rs6717960 or rs2385833). Panel D shows quantitative trait loci (QTLs) in rats coarsely mapped to humans (blood pressure QTL 34, 108, 185 and cardiac mass QTL28; source: UCSC Genome Browser) and human QTLs (cardioskeletal myopathy, 601419 and dilated myopathy, 604765; source: PUBMED OMIM database) for the region shown in (B) around rs4674485. LOC10012: Hypothetical LOC100129746; SLC4A3: Solute carrier family 4, anion exchanger, member; STK111P: Serine/threonine kinase 11 interacting protein; INHA: Inhibin, alpha; OBSL1: Obscurin-like 1;

FIG. 4 shows the genomic context for rs1466560 [G/T] (chromosome 3p26) associated with development of AF among hypertensives. Panel A shows probability of association (plotted as −log 10 p) for rs4674485 (The highest p-value indicated by vertical dotted lines) and surrounding SNPs. Panel B shows LD pattern from HapMap™-CEU in a region (dashed lines)+/−200 Kb around rs1466560 (Haploview). Panel C shows LD information for SNPs in discovery (rs1466560) and replication cohorts (rs10510266). Panel D shows QTLs in rats coarsely mapped to humans (blood pressure QTL 116; Cardiac mass QTL 18; Heart rate QTL 5; Stress response QTL 4; source: UCSC Genome Browser) and human QTLs (Hypertrophic cardiomyopathy 608751; source: PUBMED OMIM database) for the region shown in (B) around rs1466560. TRNT1: tRNA nucleotidyl transferase, CCA-adding, 1; IL5RA: Interleukin 5 receptor, alpha; CNTN4: Contactin 4; LRRN1: Leucine rich repeat neuronal 1 LOC10012: Locus100129198; LOC10013: Locus 100130207;

FIG. 5 shows ROC curves derived from logistic models containing single or combined factors including clinical (PP: pulse pressure), echocardiographic (LA vol; Left atrial volume) or neuroendocrine predictors (ANP; Atrial natriuretic peptide) with or without genetic information (SNPs: single nucleotide polymorphisms; presence or absence of any risk allele: rs4674485A or rs1466560G);

FIG. 6 shows various polymorphisms which are associated with increased risk of developing AF together with their flanking sequences. The alleles associated with an increased risk of AF are identified in bold; and

FIG. 7 shows the possible sequences of the polymorphic markers of the present invention which exclude the reference sequences not associated with a susceptibility to AF.

### DESCRIPTION OF ILLUSTRATIVE EMBODIMENTS

In order to identify new markers associated with AF, the present inventors have conducted a genome wide association analysis in a cohort of hypertensive subjects. The joint effect of genetic, clinical and neuroendocrine factors for the prediction of AF was also evaluated.

A case-control study of a hospital cohort of age- and sex-matched hypertensives vs. those without history of paroxysmal AF was conducted using 50K Affymetrix™ Genechips, and one million permutations were performed for top associated SNPs. Replication studies were performed in a larger cohort of participants in the ADVANCE study. ROC curves were constructed to compare models containing clinical (e.g., pulsed pressure and left atrial volume), neuroendocrine (e.g., ANP concentration) and/or genetic factors. Here, the present inventors report the top 10 Single Nucleotide Polymorphisms (SNPs) associated with an increased risk of suffering from AF. Two SNPs (Combined-P): rs4674485A/G (P=6.0×10⁻⁷) and rs1466560G/T (P=1.0×10⁻⁶) were selected for final modeling. After stepwise logistic regression analysis, pulse pressure, left atrial volume, atrial natriuretic peptide and the risk alleles remained as independent predictors of AF. The area under the curve for models containing only clinical factors (pulse pressure and left atrial volume) was 0.75, clinical and neuroendocrine (pulse pressure, left atrial volume and atrial natriuretic peptide) 0.87 and joint clinical, endocrine and genetic factors 0.90.

Results demonstrate that clinical, neuroendocrine and genetic factors are independently associated with AF and contribute to risk prediction. Genetic variants have an additive effect at all levels of risk determined by clinical factors.

The present invention provides additional risk stratification with genetic testing. Understanding that the individual in question carries either a risk or a protective polymorphic marker can be an invaluable contribution to diagnostic and/or treatment decision making. This way, in some cases, unnecessary testing and therapy may be avoided, and in other cases, with the help of more aggressive diagnostic approach, AF may be diagnosed and/or proper therapy initiated and later complications of disease diminished.

### DEFINITIONS

As used herein, the terms “clinical factors” refer to factors that are readily observable by a physician or other medical practitioners, without being so limited, it includes pulse pressure and left atrial volume.

As used herein, the terms “neuroendocrine factors” relate to factors involving the interaction between the nervous system and the hormones of the endocrine glands. Without being so limited, it includes concentration of atrial natriuretic peptide (ANP), serotonin, catecholamines and vasopressin.

As used herein, the terms “neurohormonal factors” comprise any hormone produced and released by neurons (includes serotonin, cathecholamine and vasopressin).

### AF

The expression or term “atrial fibrillation” or “AF” as used herein refers to atrial fibrillation (AF) as commonly defined according to established medical criteria. Generally, AF is characterized by an abnormal heart rhythm (cardiac arrhythmia) which involves the two small, upper heart chambers (the atria). Heart beat in a normal heart starts after electricity generated in the atria by the sinoatrial node spreads through the heart and causes contraction of the heart muscle and pumping of the blood. In AF, the regular electrical impulses of the sinoatrial node are replaced by disorganized, rapid electrical impulses which result in irregular heartbeat. There is a lack of unity in the function of the contracting cells of the heart muscle. AF is often asymptomatic but may result in symptoms of palpitations, exercise intolerance, fainting, shortness of breadth, chest pain or even heart failure. The erratic motion of the atria leads to blood stagnation (stasis) which increases the risk of blood clots that may travel from the heart to the brain and cause a stroke.

AF is diagnosed on an electrocardiogram, an investigation performed routinely whenever irregular heart beat is suspected. Characteristic findings include absence of P waves, unorganized electrical activity in their place and irregularity of R-R interval due to irregular conduction of impulses to the ventricles. If paroxysmal AF is suspected, episodes may be documented with the use of Holter monitoring (continuous ECG recording for 24 hours or longer).

AF includes a) paroxysmal AF which is generally defined by recurrent episodes that self terminate in less than 7 days; b) persistent AF which comprises recurrent episodes that last 7 days or more; and c) permanent AF which includes an ongoing long term episode.

### Polymorphism, Variants and SNPs

The genome exhibits sequence variability between individuals at many locations in the genome. Such variations in sequence are commonly referred to as polymorphisms, and there are many such sites within each genome. For example, the human genome exhibits sequence variations which occur on average every 500 base pairs.

The most common sequence variants consist of base variations at a single base position in the genome, and such sequence variants, or polymorphisms, are commonly called Single Nucleotide Polymorphisms (“SNPs”). These SNPs are believed to have occurred in a single mutational event, and therefore there are usually two alleles possible at each SNP site; the original allele and the mutated allele. Due to natural genetic drift and possibly also selective pressure, the original mutation has resulted in a polymorphism characterized by a particular frequency of its alleles in any given population. There may also exist SNPs that vary between paired chromosomes in an individual. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

Many other types of sequence variants are found in the human genome, including microsatellites, insertions, deletions, inversions and copy number variations. A polymorphic microsatellite has multiple small repeats of bases (such as CA repeats, TG on the complementary strand) at a particular site in which the number of repeat lengths varies in the general population.

In general terms, each version of the sequence with respect to the polymorphic site represents a specific allele of the polymorphic site. These sequence variants can all be referred to as polymorphisms occurring at specific polymorphic sites characteristic of the sequence variant in question. In general terms, polymorphisms can comprise any number of specific alleles.

In some instances, reference is made to different alleles at a polymorphic site without choosing a reference allele. Alternatively, a reference sequence can be indicated for a particular polymorphic site. The reference allele is sometimes referred to as the “wild-type” allele and it usually is chosen as either the first sequenced allele or as the allele from a “non-affected” or control/reference individual (e.g., an individual that does not display a trait or disease phenotype i.e., which does not suffer from AF).

### Polymorphic Marker

A “polymorphic marker”, also referred to as a “genetic marker”, as described herein, refers to a genomic polymorphic site. Each polymorphic marker has at least two sequence variations characteristic of particular alleles at the polymorphic site. Thus, genetic association of AF to a polymorphic marker implies that there is association to at least one specific allele of that particular polymorphic marker with AF. The marker can comprise any allele of any variant type found in the genome, including SNPs, microsatellites, insertions, deletions, duplications and translocations. The polymorphic marker, if found in a transcribed region of the genome can be detected not only in genomic DNA but also in RNA. In addition, if the polymorphism is found in the gene portion that is translated into a polypeptide or protein, the polymorphic marker can be detected at the protein/polypeptide level.

The polymorphic marker and its specific sequence variation can be detected by various means such as by sequencing the nucleic acid or protein. Alternatively, when the polymorphism affects the function of the gene or of its translated protein/polypeptide, the biological activity can be evaluated in order to identify which allele is present in the subject's sample. For example, if a particular risk allele affects the enzymatic activity of the protein, then, the presence of the allele can be assessed by performing an enzymatic test. Alternatively, if the risk allele affects the expression level of a polypeptide or nucleic acid, then, the presence of the allele can be determined by assessing the expression level (e.g., Immunoassays, amplification assays, etc.) of such protein or polypeptide and comparing it to a reference level which is considered not associated with AF.

An “allele” refers to the nucleotide sequence of a given locus (position) on a chromosome. A polymorphic marker allele thus refers to the composition (i.e., sequence) of the marker on a chromosome. Genomic DNA from an individual contains two alleles for any given polymorphic marker, representative of each copy of the marker on each chromosome. A “risk allele”, a “susceptibility allele” or a “predisposition allele” is an allele that is associated with an increased risk (i.e. compared to a control/reference) or predisposition to suffering from AF or stroke. Similarly, a risk polymorphic marker or haplotype of the present invention is one where at least one allele of at least one marker or haplotype is more frequently present in an individual at risk for the disease or trait (i.e. AF) (affected), compared to the frequency of its presence in a reference/control group, and wherein the presence of the marker or haplotype is indicative of susceptibility/predisposition to the disease (AF). Conversely, a “protective allele” is a sequence variation of a polymorphic marker that is associated with a lower risk (i.e., compared to a control/reference) or predisposition to suffering from AF or stroke.

A “haplotype” refers to a segment of DNA that is characterized by a specific combination of alleles arranged along the segment. For diploid organisms such as humans, a haplotype comprises one member of the pair of alleles for each polymorphic marker or locus. In a certain embodiment, the haplotype can comprise two or more alleles, three or more alleles, four or more alleles, five or more alleles, eight or more alleles, or 10 or more alleles, each allele corresponding to a specific polymorphic marker along the segment. Haplotypes can comprise a combination of various polymorphic markers, e.g., SNPs and microsatellites, having particular alleles at the polymorphic sites. The haplotypes thus comprise a combination of alleles at various polymorphic markers.

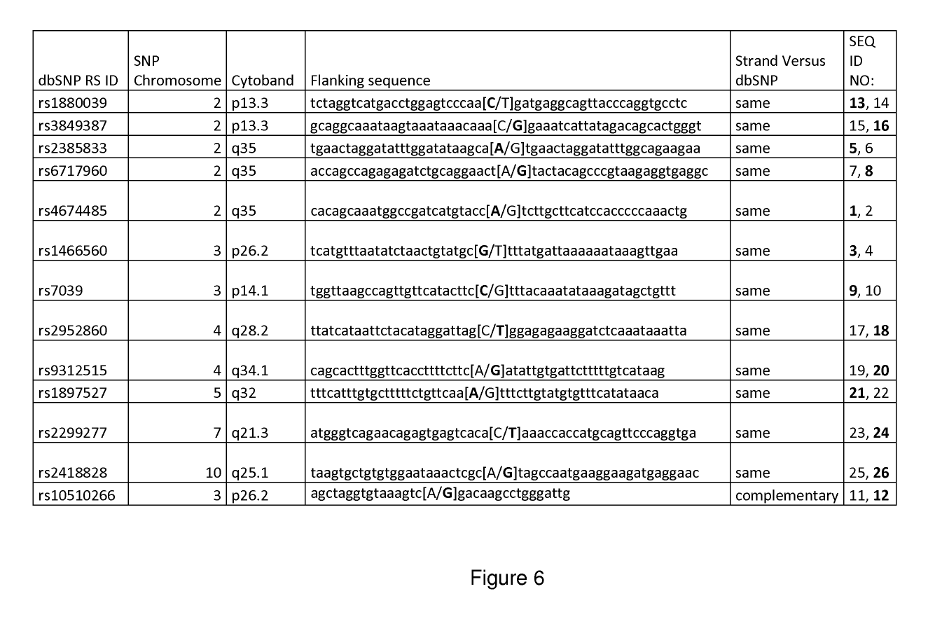

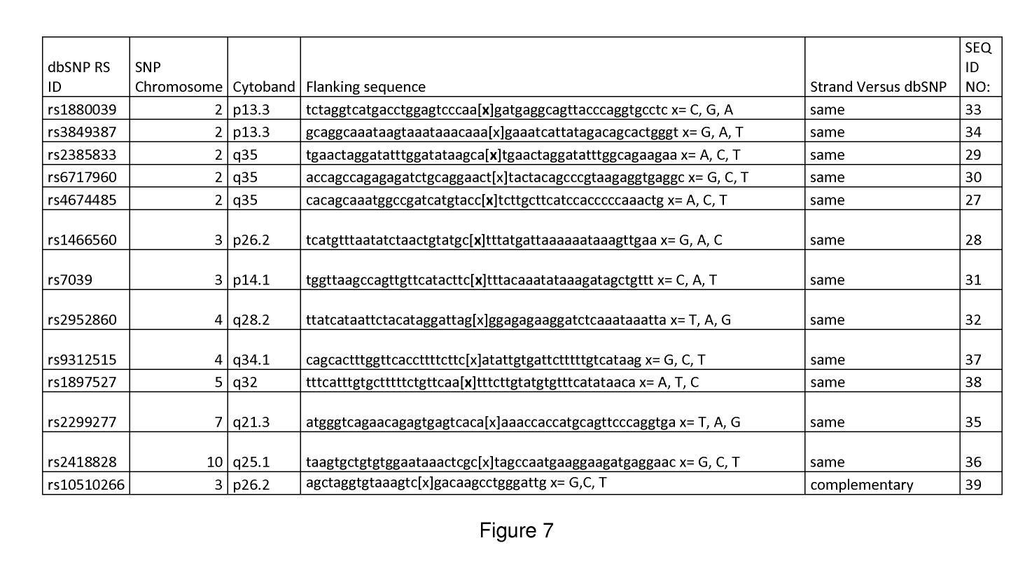

A “nucleic acid associated with AF” as used herein is a nucleic acid (e.g., genomic DNA or RNA) that comprises a polymorphic marker of the present invention, or any substitute polymorphic marker in linkage disequilibrium therewith, which is associated with AF. This nucleic acid may be of any length as long as it comprises the polymorphic region that is relevant to the determination of the presence or absence of predisposition to AF (e.g., the polymorphic markers listed in Table 4 or FIG. 6 or 7). For example, it can consist of a whole gene sequence (including the promoter or any other regulatory sequence) or of a fragment thereof. Similarly, a “polypeptide associated with AF” or a “protein associated with AF” refers to a protein or polypeptide which is encoded by a nucleic acid comprising a polymorphic marker of the present invention, or any marker in linkage disequilibrium therewith, which is associated with AF.

As used herein, the term “hypertension” refers to a chronic medical condition in which the blood pressure is elevated. It is also referred to as high blood pressure or shortened to HT. The word “hypertension”, by itself, normally refers to systemic, arterial hypertension. Without being so limited, it typically refers to a systolic blood pressure (SBP)>140 mmHg and/or diastolic blood pressure (DBP)>90 mmHg in untreated subjects.

The term “predisposition”, in the context of predisposition to AF or stroke as described herein, encompasses increased susceptibility/risk to develop AF. Thus, particular polymorphic markers and/or haplotypes of the invention constitute an indication of an increased susceptibility (predisposition, i.e., increased risk) to AF or stroke in a subject (e.g., as characterized by a relative risk (RR) or odds ratio (OR) greater than one).

The term “susceptibility”, as described herein, encompasses both increased susceptibility (predisposition) and decreased susceptibility (protection). Thus, particular polymorphic markers and/or haplotypes of the invention may be characteristic of an increased susceptibility (predisposition) (i.e., increased risk) to AF or stroke, (e.g., as characterized by a relative risk (RR) or odds ratio (OR) greater than one). Alternatively, the markers and/or haplotypes of the invention are characteristic of a decreased susceptibility (i.e., decreased risk) of AF or stroke, (e.g., as characterized by a RR or OR less than one).

The odds ratio (OR) is a measure of effect size, describing the strength of association or non-independence between two binary data values. The OR is the ratio of the odds of an event occurring in one group to the odds of it occurring in another group. These groups might be men and women, an experimental group and a control group, or any other dichotomous classification. An odds ratio of 1 indicates that the condition or event under study is equally likely to occur in both groups. An odds ratio greater than 1 indicates that the condition or event is more likely to occur in the first group.

### Sample

The term “sample” as used herein refers to any type of biological sample in which the presence of a polymorphic marker, or other markers (e.g., neuroendocrine factor or neurohormonal factor) of the present invention (at the nucleic acid level or at the protein level) may be determined, including but not limited to, blood (including plasma and serum), urine, saliva, amniotic fluid, tissue biopsy etc. The sample may be a crude sample or a purified sample, it may be a nucleic acid sample or a protein sample.

A “nucleic acid sample” is a sample that may contains nucleic acids associated with AF according to the present invention. In certain embodiments, i.e. the detection of specific polymorphic markers and/or haplotypes, the nucleic acid sample comprises genomic DNA. In other embodiments, where the polymorphic marker is located in the transcribed portion of a gene, the nucleic acid sample is RNA. Such a nucleic acid sample can be obtained from any source that contains nucleic acids, including for example a blood sample (including plasma and serum) or tissue sample from skin, muscle, buccal or conjunctival mucosa, gastrointestinal tract or other organs. Similarly, a “protein sample” or a “polypeptide sample” is a sample comprising polypeptides or proteins associated with AF in accordance with the present invention.

### Reference and Control

The terms “reference sequence” and “control sequence” are used herein interchangeably. Typically, a reference sequence is referred to for a particular sequence. Alleles that differ from the reference sequence (e.g., sequence found in an individual not suffering from AF; a sequence found in an individual not suffering from hypertension) are sometimes referred to as “variant” alleles (i.e. a sequence that is associated with AF). A variant sequence, as used herein, refers to a sequence that differs from the reference sequence but is otherwise substantially similar. Alleles of the polymorphic markers described herein are variants. Additional variants can include changes that affect a polypeptide sequence. Sequence differences, when compared to a reference nucleotide sequence, can include the insertion or deletion of a single nucleotide, or of more than one nucleotide, resulting in a frame shift; the change of at least one nucleotide, resulting in a change in the encoded amino acid; the change of at least one nucleotide, resulting in the generation of a premature stop codon; the deletion of several nucleotides, resulting in a deletion of one or more amino acids encoded by the nucleotides; the insertion of one or several nucleotides, such as by unequal recombination or gene conversion, resulting in an interruption of the coding sequence of a reading frame; duplication of all or a part of a sequence; transposition; or a rearrangement of a nucleotide sequence. Such sequence changes can alter the polypeptide encoded by the nucleic acid. For example, if the change in the nucleic acid sequence causes a frame shift, the frame shift can result in a change in the encoded amino acids, and/or can result in the generation of a premature stop codon, causing generation of a truncated polypeptide. Alternatively, a polymorphism associated with a disease or trait can be a synonymous change in one or more nucleotides (i.e., a change that does not result in a change in the amino acid sequence). Such a polymorphism can, for example, alter splice sites, affect the stability or transport of mRNA, or otherwise affect the transcription or translation of an encoded polypeptide. It can also alter DNA to increase the possibility that structural changes, such as amplifications or deletions, occur at the somatic level. The polypeptide encoded by the reference nucleotide sequence is the “reference” polypeptide with a particular reference amino acid sequence, and polypeptides encoded by variant alleles are referred to as “variant” polypeptides with variant amino acid sequences. A sequence or a reference sequence can either represent the (+) or (−) direction of double stranded DNA. Such sequences are related as being the reverse complement of one another, as well known to the skilled person.

As used herein, the terms “reference group” and control “group” are used interchangeably. The reference group may in one embodiment be a population sample, i.e. a random sample from the general population. In another embodiment, the reference group is represented by a group of individuals who are disease-free i.e., not suffering from AF or stroke. Such disease-free control may in one embodiment be characterized by the absence of one or more specific disease-associated symptoms. In an embodiment, the reference group is a group of hypertensive individuals not suffering from AF.

As used herein the terms “control sample” and “reference sample” are used interchangeably and refer to a sample isolated from a reference group or from an individual from this group. The terms “reference level” or “control level” when used in the context of a mRNA/protein/polypeptide expression or activity of a risk allele, refer to the expression level of the corresponding allele in an individual not suffering from AF or stroke, or to the average or median value obtained following determination of the corresponding allele expression or activity in a plurality of samples (e.g., samples obtained from a reference group).

### Assessment of Polymorphic Markers

Alleles for SNP markers as referred to herein refer to the bases A, C, G or T as they occur at the polymorphic site in the SNP assay employed. The person skilled in the art will realize that by assaying or reading the opposite DNA strand, the complementary allele can in each case be measured. Thus, for a polymorphic site (polymorphic marker) characterized by an A/G polymorphism, the assay employed may be designed to specifically detect the presence of one or both of the two bases possible, i.e. A and G. Alternatively, by designing an assay that is designed to detect the opposite strand on the DNA template, the presence of the complementary bases T and C can be measured. Quantitatively (for example, in terms of relative risk), identical results would be obtained from measurement of either DNA strand (+ strand or − strand).

Detecting specific polymorphic markers and/or haplotypes can be accomplished by methods known in the art for detecting sequences at polymorphic sites. For example, standard techniques for genotyping for the presence of SNPs and/or microsatellite markers can be used, such as fluorescence-based techniques (Chen, X. et al., Genome Res. 9(5): 492-98 (1999)), utilizing PCR, LCR, Nested PCR and other techniques for nucleic acid amplification. Specific methodologies available for SNP genotyping include, but are not limited to, TaqMann™ genotyping assays and SNPlex™ platforms (Applied Biosystems), mass spectrometry (e.g., MassARRAY™ system from Sequenom), minisequencing methods, real-time PCR, Bio-Plex™ system (BioRad), CEQ and SNPstream™ systems (Beckman), Molecular Inversion Probew array technology (e.g., Affymetrix GeneChip), and BeadArray™ Technologies (e.g., Illumina GoldenGate and Infinium assays). By these or other methods available to the person skilled in the art, one or more alleles at polymorphic markers, including microsatellites, SNPs or other types of polymorphic markers, can be identified.

In certain methods described herein, an individual who is predisposed (i.e., increased risk) to AF, is an individual in whom at least one specific allele at one or more polymorphic marker or haplotype conferring increased susceptibility (i.e. compared to the general population or compared to hypertensive individuals not at risk of developing AF) for AF is identified (i.e., risk marker alleles or haplotypes). In one aspect, the risk marker or haplotype is one that confers a significant increased risk (or predisposition) for AF. In an embodiment, significance associated with a marker or haplotype is measured by an odds ratio (OR). In a further embodiment, the significance is measured by a percentage. In one embodiment, a significant increased risk is measured as a risk (relative risk and/or odds ratio) of at least 1.2, at least 2, at least 3, at least 4, at least 5, and at least 5.5 as compared to that in the general population. In a particular embodiment, a risk (relative risk and/or odds ratio) is significant if the value OR=1.0 is not included in the 95% confidence interval. In another particular embodiment, a risk of at least 1.3 is significant. In yet another embodiment, a risk of at least 1.4 is significant. In a further embodiment, a relative risk of at least about 1.5 is significant. In another further embodiment, a significant increase in risk is at least about 2.4 is significant. However, other cutoffs are also contemplated, e.g. at least 1.15, 1.25, 1.35, 2, 2.5, 3, 3.5, 4, 4.5, 5, 5.5, 6 and so on, and such cutoffs are also within scope of the present invention. In other embodiments, a significant increase in risk is at least about 20%, including but not limited to about 25%, 30%, 35%, 40%, 45%, 50%, 55%, 60%, 65%, 70%, 75%, 80%, 85%, 90%, 95%, 100%, 150%, 200%, 300%, and 500%. In one particular embodiment, a significant increase in risk is at least 20%. In other embodiments, a significant increase in risk is at least 30%, at least 40%, at least 50%, at least 60%, at least 70%, at least 80%, at least 90% and at least 100%. Other cutoffs or ranges as deemed suitable by the person skilled in the art to characterize the invention are however also contemplated, and those are also within scope of the present invention.

In a preferred embodiment, the method of the present invention are designed to be at least 75% specific, more preferably 80% specific, even more preferably 85% specific. Specificity measures the proportion of negatives which are correctly identified (e.g. the percentage of people who are correctly identified as not being susceptible to AF or who will not develop AF). Specificity is the number of true negatives divided by the number of true negative+the number of false positive. A specificity of 100% means that the test recognizes all actual negatives i.e., that all non susceptible people will be recognized as such. Because 100% specificity means no positives are erroneously tagged, a positive result in a high specificity test is used to confirm the susceptibility to the disease i.e., the susceptibility to AF.

### Nucleic Acids

The nucleic acid molecules (e.g., oligonucleotides, primers, probes) of the present invention have a variety of uses, especially in assessing the risk or predisposition of developing AF and/or stroke. For example, the nucleic acid molecules are useful as hybridization probes, such as for genotyping SNPs in messenger RNA, transcript, cDNA, genomic DNA, amplified DNA or other nucleic acid molecules, and for isolating full-length cDNA and genomic clones.

A probe can hybridize to a SNP-containing target sequence in a sequence-specific manner such that it distinguishes the target sequence from other nucleotide sequences which vary from the target sequence only by which nucleotide is present at the SNP site. Such a probe is particularly useful for detecting the presence of a SNP-containing nucleic acid in a test sample, or for determining which nucleotide (allele) is present at a particular SNP site (i.e., genotyping the SNP site).

A nucleic acid hybridization probe may be used for determining the presence, level, form, and/or distribution of nucleic acid expression. The nucleic acid whose level is determined can be DNA or RNA. Accordingly, probes specific for the SNPs described herein can be used to assess the presence, expression and/or gene copy number in a given cell, tissue, or organism. In vitro techniques for detection of mRNA include, for example, Northern blot hybridizations and in situ hybridizations. In vitro techniques for detecting DNA include Southern blot hybridizations and in situ hybridizations (Sambrook and Russell, 2000, Molecular Cloning: A Laboratory Manual, Cold Spring Harbor Press, Cold Spring Harbor, N.Y.). Thus, the nucleic acid molecules of the invention can be used as hybridization probes to detect the SNPs disclosed herein, thereby determining whether an individual with one or more of the polymorphism of the present invention is at risk (or predisposed) for developing AF.

Nucleic acid samples can be genotyped to determine which allele(s) is/are present at any given genetic region (e.g., SNP position) of interest by methods well known in the art. The neighboring sequence can be used to design SNP detection reagents such as oligonucleotide probes, which may optionally be implemented in a kit format.

SNP genotyping can include the steps of, for example, collecting a biological sample from a human subject (e.g., sample of tissues, cells, fluids (e.g., saliva, blood, mucus), secretions, etc.), isolating nucleic acids (e.g., genomic DNA, mRNA or both) from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/or amplification will only occur if a particular SNP allele is present or absent). In some assays, the size of the amplification product is detected and compared to the length of a control sample; for example, deletions and insertions can be detected by a change in size of the amplified product compared to a normal genotype.

A primer or probe of the present invention is typically at least about 8 nucleotides in length. In one embodiment of the invention, a primer or a probe is at least about 10 nucleotides in length. In a preferred embodiment, a primer or a probe is at least about 12 nucleotides in length. In a more preferred embodiment, a primer or probe is at least about 16, 17, 18, 19, 20, 21, 22, 23, 24 or 25 nucleotides in length. While the maximal length of a probe can be as long as the target sequence to be detected, depending on the type of assay in which it is employed, it is typically less than about 50, 60, 65, or 70 nucleotides in length. In the case of a primer, it is typically less than about 30 nucleotides in length. In a specific preferred embodiment of the invention, a primer or a probe is within the length of about 18 and about 28 nucleotides. However, in other embodiments, such as nucleic acid arrays and other embodiments in which probes are affixed to a substrate, the probes can be longer, such as on the order of 30-70, 75, 80, 90, 100, or more nucleotides in length (see the Section entitled “Kits”).

For analyzing SNPs, it may be appropriate to use oligonucleotides specific for alternative SNP alleles. Such oligonucleotides that detect single nucleotide variations in target sequences may be referred to by such terms as “allele-specific oligonucleotides”, “allele-specific probes”, or “allele-specific primers”. The design and use of allele-specific probes for analyzing polymorphisms is described in, e.g., Mutation Detection A Practical Approach, ed. Cotton et al. Oxford University Press, 1998; Saiki et al., Nature 324, 163-166 (1986); Dattagupta, EP235.726; and Saiki, WO 89/11548.

While the design of each allele-specific primer or probe depends on variables such as the precise composition of the nucleotide sequences flanking a SNP position in a target nucleic acid molecule, and the length of the primer or probe, another factor in the use of primers and probes is the stringency of the conditions under which the hybridization between the probe or primer and the target sequence is performed. Higher stringency conditions utilize buffers with lower ionic strength and/or a higher reaction temperature, and tend to require a more perfect match between probe/primer and a target sequence in order to form a stable duplex. If the stringency is too high, however, hybridization may not occur at all. In contrast, lower stringency conditions utilize buffers with higher ionic strength and/or a lower reaction temperature, and permit the formation of stable duplexes with more mismatched bases between a probe/primer and a target sequence. By way of example and not limitation, exemplary conditions for high stringency hybridization conditions using an allele-specific probe are as follows: Prehybridization with a solution containing 5× standard saline phosphate EDTA (5×SSPE), 0.5% NaDodSO₄ (SDS) at 55° C., and incubating probe with target nucleic acid molecules in the same solution at the same temperature, followed by washing with a solution containing 2×. SSPE, and 0.1% SDS at 55° C. or room temperature.

Moderate stringency hybridization conditions may be used for allele-specific primer extension reactions with a solution containing, e.g., about 50 mM KCI at about 46° C. Alternatively, the reaction may be carried out at an elevated temperature such as 60° C. In another embodiment, a moderately stringent hybridization condition suitable for oligonucleotide ligation assay (OLA) reactions wherein two probes are ligated if they are completely complementary to the target sequence may utilize a solution of about 100 mM KCl at a temperature of 46° C.

In a hybridization-based assay, allele-specific probes can be designed that hybridize to a segment of target DNA from one individual but do not hybridize to the corresponding segment from another individual due to the presence of different polymorphic forms (e.g., alternative SNP alleles/nucleotides) in the respective DNA segments from the two individuals. Hybridization conditions should be sufficiently stringent that there is a significant detectable difference in hybridization intensity between alleles, and preferably an essentially binary response, whereby a probe hybridizes to only one of the alleles or significantly more strongly to one allele. While a probe may be designed to hybridize to a target sequence that contains a SNP site such that the SNP site aligns anywhere along the sequence of the probe, the probe is preferably designed to hybridize to a segment of the target sequence such that the SNP site aligns with a central position of the probe (e.g., a position within the probe that is at least three nucleotides from either end of the probe). This design of probe generally achieves good discrimination in hybridization between different allelic forms.

In another embodiment, a probe or primer may be designed to hybridize to a segment of target DNA such that the SNP aligns with either the 5′ most end or the 3′ most end of the probe or primer.

Oligonucleotide probes and primers may be prepared by methods well known in the art. Chemical synthetic methods include, but are limited to, the phosphotriester method described by Narang et al., 1979, Methods in Enzymology 68:90; the phosphodiester method described by Brown et al., 1979, Methods in Enzymology 68:109, the diethylphosphoamidate method described by Beaucage et al., 1981, Tetrahedron Letters 22: 1859; and the solid support method described in U.S. Pat. No. 4,458,066.

Allele-specific probes are often used in pairs (or, less commonly, in sets of 3 or 4, such as if a SNP position is known to have 3 or 4 alleles, respectively, or to assay both strands of a nucleic acid molecule for a target SNP allele), and such pairs may be identical except for a one nucleotide mismatch that represents the allelic variants at the SNP position.

Commonly, one member of a pair perfectly matches a reference form of a target sequence that has a more common SNP allele (i.e., the allele that is more frequent in the target population) and the other member of the pair perfectly matches a form of the target sequence that has a less common SNP allele (i.e., the allele that is rarer in the target population, for example the risk allele). In the case of an array, multiple pairs of probes can be immobilized on the same support for simultaneous analysis of multiple different polymorphisms.

In one type of PCR-based assay, an allele-specific primer hybridizes to a region on a target nucleic acid molecule that overlaps a SNP position and only primes amplification of an allelic form to which the primer exhibits perfect complementarity (Gibbs, 1989, Nucleic Acid Res. 17 2427-2448). Typically, the primer's 3′-most nucleotide is aligned with and complementary to the SNP position of the target nucleic acid molecule. This primer is used in conjunction with a second primer that hybridizes at a distal site. Amplification proceeds from the two primers, producing a detectable product that indicates which allelic form is present in the test sample. A control is usually performed with a second pair of primers, one of which shows a single base mismatch at the polymorphic site and the other of which exhibits perfect complementarity to a distal site. The single-base mismatch prevents amplification or substantially reduces amplification efficiency, so that either no detectable product is formed or it is formed in lower amounts or at a slower pace. The method generally works most effectively when the mismatch is at the 3′-most position of the oligonucleotide (i.e., the 3′-most position of the oligonucleotide aligns with the target SNP position) because this position is most destabilizing to elongation from the primer.

### Linkage Disequilibrium

The natural phenomenon of recombination, which occurs on average once for each chromosomal pair during each meiotic event, represents one way in which nature provides variations in sequence (and biological function by consequence). It has been discovered that recombination does not occur randomly in the genome; rather; there are large variations in the frequency of recombination rates, resulting in small regions of high recombination frequency (also called recombination hotspots) and larger regions of low recombination frequency, which are commonly referred to as Linkage Disequilibrium (LD) blocks (Myers, S. et al., Biochem Soc Trans 34:526-530 (2006); Jeffreys, A J., et al., Nature Genet 29:217-222 (2001); May, C. A., et al., Nature Genet 31:272-275 (2002)).

LD refers to a non-random assortment of two genetic elements. Thus, the term LD refers to a non-random genetic association between one or more allele(s) of two different polymorphic markers, that is due to the physical proximity of the two loci. LD is present when two DNA segments that are very close to each other on a given chromosome will tend to remain unseparated for several generations with the consequence that alleles of a polymorphic marker in one segment will show a non-random association with the alleles of a different polymorphic marker located in the other DNA segment nearby. As demonstrated herein, testing of a polymorphic marker (e.g., rs2385833 or rs6717960) (indirect testing), in LD with another polymorphic marker of the present invention (e.g., rs4674485) will give equivalent information as testing for the rs4674485 polymorphism directly. Similarly, testing for rs10510266 will give equivalent information as testing for the rs1466560 polymorphism directly. Hence, rs2385833 and rs6717960 are in LD with rs4674485, and rs10510266 is in LD with rs1466560. This situation is encountered throughout the human genome when two DNA polymorphisms that are very close to each other are studied. LD and, the use thereof in inheritance studies is well known in the art to which the present invention pertains as exemplified by publications such as Risch and Merikangas, Science 273: 1516-1517 (1996); Maniatis, Methods Mol Biol. 376: 109-21 (2007) and Borecki et al., Adv Genet 60: 51-74 (2008).

For example, if a particular genetic element (e.g., an allele of a polymorphic marker, or a haplotype) occurs in a population at a frequency of 0.25 (25%) and another element occurs at a frequency of 0.25 (25%), then the predicted occurrence of a person having both elements is 0.125 (12.5%), assuming a random distribution of the elements. However, if it is discovered that the two elements occur together at a frequency higher than 0.125, then the elements are said to be in LD, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurrence of each allele or haplotype in the population.

Many different measures have been proposed for assessing the strength of LD. Most capture the strength of association between pairs of biallelic sites. Two important pairwise measures of LD are r² (sometimes denoted Δ) and |D′|. Both measures range from 0 (no disequilibrium) to 1 (complete disequilibrium), but their interpretation is slightly different. |D′| is defined in such a way that it is equal to 1 if just two or three of the possible haplotypes are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 1 if only two haplotypes are present.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model).

One approach of measuring LD across a region is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots. For the methods and procedures described herein, a significant r² value can be at least 0.1 such as at least 0.1, 0.2, 0.3, 0.4, 0.5, 0.6, 0.7, 0.75, 0.8, 0.85, 0.9, 0.91, 0.92, 0.93, 0.94, 0.95, 0.96, 0.97, 0.98, 0.99 or 1.0. In one preferred embodiment, the significant r² value can be at least 0.8. Alternatively, linkage disequilibrium as described herein, refers to linkage disequilibrium characterized by values of |D′| of at least 0.2, such as 0.3, 0.4, 0.5, 0.6, 0.7, 0.8, 0.85, 0.9, 0.95, 0.96, 0.97, 0.98 or 0.99. Thus, LD represents a correlation between alleles of distinct polymorphic markers. It is measured by correlation coefficient or |D′| (r² up to 1.0 and |D′| up to 1.0). LD can be determined in a single human population, as defined herein, or it can be determined in a collection of samples comprising individuals from more than one human population. In one embodiment of the invention, LD is determined in a sample from one or more of the HapMap™ populations (Caucasian, African, Japanese, Chinese), as defined (http://www.hapmap.org). In one such embodiment, LD is determined in the CEU population of the HapMap™ III samples.

It is now established that many portions of the human genome can be broken into series of discrete haplotype blocks containing a few common haplotypes; for these blocks, LD data provides little evidence indicating recombination (see, e.g., Wall., J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294: 1719-1723 (2001); Dawson, E. et al., Nature 4JS:544-548 (2002); Phillips, M. S. et al., Nature Genet 33:382-387 (2003)).

There are two main methods for defining these haplotype blocks: Blocks can be defined as regions of DNA that have limited haplotype diversity (see, e.g., Daly, M. et al., Nature Genet. 29:229-232 (2001); Patil, N. et al., Science 294: 1719-1723 (2001); Dawson, E. et al., Nature 418:544-548 (2002); Zhang, K. et al., Proc. Natl. Acad. Sci. USA 99:7335-7339 (2002)), or as regions between transition zones having extensive historical recombination, identified using LD (see, e.g., Gabriel, S. B. et al., Science 296:2225-2229 (2002); Phillips, M. S. et al., Nature Genet. 33:382-387 (2003); Wang, N. et al., Am. J. Hum. Genet. 7:1227-1234 (2002); Stumpf, M. P., and Goldstein, D. B., Curr. Biol. 13: 1-8 (2003)). More recently, a fine-scale map of recombination rates and corresponding hotspots across the human genome has been generated (Myers, S., et al., Science 310:321-32324 (2005); Myers, S. et al., Biochem Soc Trans 34:526530 (2006)). The map reveals the enormous variation in recombination across the genome, with recombination rates as high as 10-60 cM/Mb in hotspots, while closer to 0 in intervening regions, which thus represent regions of limited haplotype diversity and high LD. The map can therefore be used to define haplotype blocks/LD blocks as regions flanked by recombination hotspots. As used herein, the terms “haplotype block” or “LD block” includes blocks defined by any of the above described characteristics, or other alternative methods used by the person skilled in the art to define such regions.

Haplotype blocks can be used to map associations between phenotype and haplotype status, using single markers or haplotypes comprising a plurality of markers.

The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist LD among the haplotype blocks.

As described herein, certain polymorphic markers and haplotypes comprising such markers are useful for risk assessment of AF. Risk assessment can involve the use of the markers for diagnosing a susceptibility/predisposition to AF. Particular alleles of polymorphic markers are found more frequently in individuals with AF, than in individuals without diagnosis of AF. Therefore, these marker alleles have predictive value for detecting a susceptibility/predisposition to AF in subjects.

Substitute polymorphic markers within haplotype blocks or LD blocks comprising risk markers, such as the markers of the present invention, can be used as substitutes for other markers and/or haplotypes within the haplotype block or LD block. Markers with values of r² equal to 1 are perfect substitutes for the risk variants, i.e. genotypes for one marker perfectly predicts genotypes for the other. Markers with smaller values of r² than 1 can also be surrogates for the risk polymorphic markers described herein, or alternatively represent variants with relative risk values as high or possibly even higher than the risk polymorphic markers of the present invention. The present invention encompasses the assessment of such substitute (surrogate) markers for the polymorphic markers of the present invention. Such markers are annotated, mapped and listed in public databases, as is well known to the skilled person, or can alternatively be identified by sequencing the region or a part of the region identified by the polymorphic markers of the present invention in a group of individuals, and identify polymorphisms in the resulting group of sequences. As a consequence, the person skilled in the art can without undue experimentation genotype substitute polymorphic markers in LD with the polymorphic markers as described herein. The substitute polymorphic markers in LD with the risk polymorphic marker detected, also have predictive value for detecting susceptibility to AF in a subject.

Thus, in one embodiment of the invention, a plurality of variants (markers and/or haplotypes) is used for overall risk assessment. These variants are in one embodiment selected from the variants as disclosed herein. Other embodiments include the use of the variants of the present invention in combination with other variants known to be useful for diagnosing a susceptibility/predisposition to AF and/or stroke.

### Treatment and Prevention of AF and Personalized Medicine

Due its temporary nature, paroxysmal AF can be very difficult to diagnose. The opportunity to diagnose the arrhythmia, institute recommended therapy, and possibly prevent a debilitating first or recurrent stroke may be missed with devastating results to the patient. Understanding that the individual in question carries either a risk or a protective polymorphic marker can be an invaluable contribution to diagnostic and/or treatment decision making. This way, in some cases, unnecessary testing and therapy may be avoided, and in other cases, with the help of more aggressive diagnostic approach, the arrhythmia may be diagnosed and/or proper therapy initiated and later complications of disease diminished.

Treatment of AF has two main objectives: (i) prevent temporary circulatory instability; and (ii) prevent stroke. The most common methods for achieving the former includes rate and rhythm control, while anticoagulation is usually the desired method for the latter. Common methods for rate control, i.e. for reducing heart rate to normal, include beta blockers, cardiac glycosides (e.g., digoxi) and calcium channel blockers (e.g., verapamil). All these medications work by slowing down the generation of pulses from the atria, and the conduction from the atria to the ventricles. Other drugs commonly used include quinidine, flecainide, propafenone, disopyramide, sotalol and amiodarone. Rhythm control can be achieved by electrical cardioversion, i.e. by applying DC electrical shock, or by chemical cardioversion, using drugs such as amiodarione, propafenone and flecainide.

Preventive measures for stroke include anticoagulants. Representative examples of anticoagulant agents are daiteparin (e.g., Fragmin), danaparoid (e.g., Orgaran), enoxaparin (e.g., Lovenox), heparin (various), tinzaparin (e.g., Innohep) and warfarin (e.g., Coumadin). Some patients with lone AF are sometimes treated with aspirin or clopidogrel. The new anticoagulant ximelagatran has been shown to prevent stroke with equal efficacy as warfarin, without the difficult monitoring process associated with warfarin and with possibly fewer adverse hemorrhagic events.

It has repeatedly been shown that therapy with warfarin anticoagulation can significantly reduce the risk of first or further episodes of stroke in the setting of AF. Therefore, anticoagulation with warfarin is standard therapy for almost all patients with AF for stroke-prevention, whether they have the permanent or paroxysmal type. The only patients for whom warfarin is not strongly recommended are those younger than 65 years old who are considered low-risk, i.e., they have no organic heart disease, including, neither hypertension no coronary artery disease, no previous history of stroke or transient ischemic attacks and no diabetes. This group has a lower risk of stroke and stroke-prevention with aspirin is recommended.

Determining who should and should not receive anti-coagulation with warfarin is not straightforward. The underlying problem is that if a subject has a yearly risk of stroke that is less than 2%, then the risks associated with taking warfarin outweigh the risk of experiencing a stroke. AF can sometimes be controlled with treatment. The natural tendency of AF, however, is to become a chronic condition. Chronic AF leads to an increased risk of death. Patients with AF have a significantly increased risk of stroke.

Accordingly, in an embodiment, the methods of the present invention further comprises determining the appropriate preventive treatment or therapy.

The person skilled in the art will appreciate and understand that the polymorphic markers of the present invention in general do not, by themselves, provide an absolute identification of individuals who will develop AF and/or indirectly who will suffer from a stroke. The polymorphic markers described herein do however indicate an increased likelihood (or decreased likelihood in the case of a protective variant) that individuals carrying the risk (or protective) variants of the invention will develop AF including symptoms associated with AF. This information is extremely valuable in itself, as it can be used to, for example, initiate preventive measures at an early stage, perform regular physical and/or mental exams to monitor the progress and/or appearance of symptoms, or to schedule exams at a regular interval to identify the condition in question, so as to be able to apply treatment at an early stage.

The knowledge about a genetic variant that confers a risk of developing AF offers the opportunity to apply a genetic test to distinguish between subjects with increased risk of developing the disease (i.e. carriers of the risk polymorphic markers) from those without an increased risk, and eventually, from those with a decreased risk of developing the disease (i.e. carriers of a protective polymorphic marker). The values of genetic testing include the ability to diagnose a predisposition to AF at an early stage, and provide this information to the clinician in order to select the most appropriate treatment, including a preventive treatment such as those discussed above.

Subjects with a family history of AF and carriers of risk polymorphic markers may benefit from genetic testing since the knowledge of the presence of a genetic risk factor, may provide increased incentive for implementing a healthier lifestyle, by avoiding or minimizing known environmental risk factors for AF. Genetic testing of AF patients may furthermore provide valuable information about the primary cause of the disease and can assist the clinician in selecting the best treatment options and medication for each individual.

Thus in an embodiment, the present invention relates to risk assessment for AF. The polymorphic markers of the present invention can be used alone (individually) or in combinations of two or more markers, as well as in combination with other factors, including other genetic risk factors or biomarkers, for risk assessment of an individual for AF. Non-limiting examples of other factors include those described herein such as pulse pressure, left atrial volume and ANP concentration as well other polymorphic markers or haplotypes identified as increasing the risk to AF (e.g., genetic variants on chromosomes 4 (e.g., 4q25), 11, 13, 15 and 16).

### Kits

As indicated above, the kits of the present invention may comprise any reagent (e.g., oligonucleotide probes, primers, antibodies, ligands, substrates, etc.) useful for determining the presence in a sample from a subject of at least one allele of at least one polymorphic marker of the present invention. Thus, in an embodiment the present invention provides a kit for determining a predisposition to atrial fibrillation (AF) in a subject comprising: reagents for determining the presence of at least one copy of a risk allele from at least one polymorphic marker in a sample from the subject, wherein the presence of at least one copy of the risk allele is indicative of a predisposition to AF and wherein said at least one polymorphic marker is: specifically a) rs4674485; b) rs1466560; c) rs1880039; d) rs3849387; e) rs7039; f) rs2952860; g) rs9312515; h) rs1897527; i) rs2299277; j) rs2418828; k) rs2385833; l) rs6717960; m) rs10510266; or n) a substitute polymorphic marker in linkage disequilibrium with any one of the polymorphic markers of a) to m).

The kits may further comprise, reagents for determining the ANP blood concentration in the same or a different sample from the subject.

Thus, the kits of the present invention may comprise as reagents, components useful in any of the methods described herein, including for example, primers, hybridization probes, allele-specific oligonucleotides, restriction enzymes (e.g., for RFLP analysis) and/or antibodies that bind to a mutated polypeptide (polymorphic polypeptide) which is encoded by a nucleic acid comprising a polymorphic marker of the present invention, reagents for amplification of nucleic acid associated with AF, reagents for analyzing the nucleic acid sequence of a nucleic acid associated with AF, means for analyzing the amino acid sequence of a polypeptide encoded by a nucleic acid associated with AF. The kit may also include any necessary buffers, enzymes (e.g., DNA polymerase) and/or reagents necessary for the allele specific detection of the polymorphic markers of the present invention. The kit may also comprise one or more labeled nucleic acids capable of allele-specific detection of one or more polymorphic markers of the present invention or any markers in linkage disequilibrium therewith as well as reagents for the detection of the label. Suitable labels are well known in the art and will be chosen according to the specific method used. Non-limiting examples of suitable labels include a radioisotope, a fluorescent label, a magnetic label, an enzyme, etc.

In particular embodiments, the kit of the present invention includes reagents for the detection of one, two, three, four, five, six, seven, eight, nine or ten or more polymorphic markers of the present invention and/or any number of polymorphic markers in LD therewith. Kits of the present invention may also comprise instructions on how to use the reagents of the kits for the methods of the present invention and how to use additional markers of predisposition to AF for these methods (e.g., pulse pressure and left atrial volume).

A person skilled in the art will recognize that, based on the SNP and associated sequence information disclosed herein, detection reagents can be developed and used to assay any SNP of the present invention individually or in combination, and such detection reagents can be readily incorporated into one of the established kit or system formats which are well known in the art. The terms “kits” or “kit”, as used herein in the context of SNP detection reagents, are intended to refer to such things as combinations of multiple SNP detection reagents, or one or more SNP detection reagents in combination with one or more other types of elements or components (e.g., other types of biochemical reagents, containers, packages such as packaging intended for commercial sale, substrates to which SNP detection reagents are attached, electronic hardware components, etc.).

Accordingly, the present invention further provides SNP detection kits, including but not limited to, packaged probe and primer sets (e.g., TaqMan probe/primer sets), arrays/microarrays of nucleic acid molecules, and beads that contain one or more probes, primers, or other detection reagents for detecting one or more SNPs of the present invention. The kit of the present invention can optionally include various electronic hardware components; for example, arrays (“DNA chips”) and micro fluidic systems (“lab-on-a-chip” systems) provided by various manufacturers typically comprise hardware components. Other kits (e.g., probe/primer sets) may not include electronic hardware components, but may be comprised of, for example, one or more SNP detection reagents (along with, optionally, other biochemical reagents) packaged in one or more containers.

In an embodiment, a SNP detection kit typically contains one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction, such as amplification and/or detection of a SNP-containing nucleic acid molecule. A kit may further contain means for determining the amount of a target nucleic acid, and means for comparing the amount with a standard, and can comprise instructions for using the kit to detect the SNP-containing nucleic acid molecule of interest. In one embodiment of the present invention, kits are provided which contain the necessary reagents to carry out one or more assays to detect one or more SNPs disclosed herein. In an embodiment of the present invention, SNP detection kits are in the form of nucleic acid arrays, or compartmentalized kits, including micro fluidic/lab-on-a-chip systems.

SNP detection kits may contain, for example, one or more probes, or pairs of probes, that hybridize to a nucleic acid molecule at or near each target SNP position. Multiple pairs of allele-specific probes may be included in the kit/system to simultaneously assay large numbers of SNPs, at least one of which is a SNP of the present invention. In some kits, the allele-specific probes are immobilized to a substrate such as an array or bead.

The terms “arrays”, “microarrays”, and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate. In one embodiment, the microarray is prepared and used according to the methods described in U.S. Pat. No. 5,837,832, Chee et al, PCT application WO95/11995 (Chee et al), Lockhart, D. J. et al. (1996; Nat. Biotech. 14: 1675-1680) and Schena, M. et al. (1996; Proc. Natl. Acad. Sci. 93: 10614-10619), all of which are incorporated herein in their entirety by reference. In other embodiments, such arrays are produced by the methods described by Brown et al., U.S. Pat. No. 5,807,522.

Nucleic acid arrays are reviewed in the following references: Zammatteo et al., “New chips for molecular biology and diagnostics”, Biotechnol Annu Rev. 2002; 8:85-101; Sosnowski et al., “Active microelectronic array system for DNA hybridization, genotyping and pharmacogenomic applications”, Psychiatr Genet. 2002 December; 12(4): 181-92; Heller, “DNA microarray technology: devices, systems, and applications”, Annu Rev Biomed Eng. 2002; 4: 129-53. Epub 2002 Mar. 22; Kolchinsky et al., “Analysis of SNPs and other genomic variations using gel-based chips”, Hum Mutat. 2002 April; 19(4):343-60; and McGall et al., “High-density genechip oligonucleotide probe arrays”, Adv Biochem Eng Biotechnol. 2002; 77:2142.

Any number of probes, such as allele-specific probes, may be implemented in an array, and each probe or pair of probes can hybridize to a different SNP position. In the case of polynucleotide probes, they can be synthesized at designated areas (or synthesized separately and then affixed to designated areas) on a substrate using a light-directed chemical process. Each DNA chip can contain, for example, thousands to millions of individual synthetic polynucleotide probes arranged in a grid-like pattern and miniaturized (e.g., to the size of a dime). Preferably, probes are attached to a solid support in an ordered, addressable array.

A microarray can be composed of a large number of unique, single-stranded polynucleotides, usually either synthetic antisense polynucleotides or fragments of cDNAs, fixed to a solid support. Typical polynucleotides are preferably about 6-60 nucleotides in length, more preferably about 15-30 nucleotides in length, and mostpreferably about 18-25 nucleotides in length. For certain types of microarrays or other detection kits, it may be preferable to use oligonucleotides that are only about 7-20 nucleotides in length. In other types of arrays, such as arrays used in conjunction with chemiluminescent detection technology, preferred probe lengths can be, for example, about 15-80 nucleotides in length, preferably about 50-70 nucleotides in length, more preferably about 55-65 nucleotides in length, and most preferably about 60 nucleotides in length. The microarray or detection kit can contain polynucleotides that cover the known 5′ or 3′ sequence of a gene/transcript or target SNP site, sequential polynucleotides that cover the full-length sequence of a gene/transcript; or unique polynucleotides selected from particular areas along the length of a target gene/transcript sequence, particularly areas corresponding to one or more SNPs. Polynucleotides used in the microarray or detection kit can be specific to a SNP or SNPs of interest (e.g., specific to a particular SNP allele at a target SNP site, or specific to particular SNP alleles at multiple different SNP sites), or specific to a polymorphic gene/transcript or genes/transcripts of interest. Hybridization assays based on polynucleotide arrays rely on the differences in hybridization stability of the probes to perfectly matched and mismatched target sequence variants. For SNP genotyping, it is generally preferable that stringency conditions used in hybridization assays are high enough such that nucleic acid molecules that differ from one another at as little as a single SNP position can be differentiated (e.g., typical SNP hybridization assays are designed so that hybridization will occur only if one particular nucleotide is present at a SNP position, but will not occur if an alternative nucleotide is present at that SNP position). Such high stringency conditions may be preferable when using, for example, nucleic acid arrays of allele-specific probes for SNP detection. Such high stringency conditions are described in the preceding section, and are well known to those skilled in the art and can be found in, for example, Current Protocols in Molecular Biology, John Wiley & Sons, N.Y. (1989), 6.3.1-6.3.6.

Using such arrays or other kits, the present invention provides methods of identifying the SNPs disclosed herein in a test sample. Such methods typically involve incubating a test sample of nucleic acids with an array comprising one or more probes corresponding to at least one SNP position of the present invention, and assaying for binding of a nucleic acid from the test sample with one or more of the probes. Conditions for incubating a SNP detection reagent (or a kit/system that employs one or more such SNP detection reagents) with a test sample vary. Incubation conditions depend on suchfactors as the format employed in the assay, the detection methods employed, and the type and nature of the detection reagents used in the assay. One skilled in the art will recognize that any one of the commonly available hybridization, amplification and array assay formats can readily be adapted to detect the SNPs disclosed herein.

A SNP detection kit/system of the present invention may include components that are used to prepare nucleic acids from a test sample for the subsequent amplification and/or detection of a SNP-containing nucleic acid molecule. Such sample preparation components can be used to produce nucleic acid extracts (including DNA and/or RNA), proteins or membrane extracts from any bodily fluids (such as blood, serum, plasma, urine, saliva, phlegm, gastric juices, semen, tears, sweat, etc.), skin, hair, cells (especially nucleated cells), biopsies, buccal swabs or tissue specimens. The test samples used in the above-described methods will vary based on such factors as the assay format, nature of the detection method, and the specific tissues, cells or extracts used as the test sample to be assayed. Methods of preparing nucleic acids, proteins, and cell extracts are well known in the art and can be readily adapted to obtain a sample that is compatible with the system utilized.

The present invention is illustrated in further details by the following non-limiting examples.

### Example 1

### Patients Material and Methods

The discovery cohort comprised 169 individuals: 148 patients with a validated history of hypertension, in sinus rhythm at the time of the study: 74 with one or more previous episodes of paroxysmal AF documented on ECG (HT+AF) and 74 individuals without any previous known episode of AF (HT). Moreover, 21 normotensive individuals without history of AF were also enrolled and used as reference for several clinical, metabolic and neurohormonal assessments. Patients were recruited in 3 clinical centers, and each center ethics committee approved the protocol of study. All participants gave their written informed consent to participate in the study.

The exclusion criteria were the presence of secondary causes of hypertension, the presence of AF and other atrial arrhythmias at the time of the study, valvular heart disease, heart failure, coronary artery disease, hepatic, renal, thyroid or lung diseases. All participants underwent physical examination and blood sample collection for lipids and neurohormonal factors. A 12-lead ECG and a transthoracic M-mode and two-dimensional Doppler echocardiograms were recorded on the day of examination.

Hypertension was defined as a systolic blood pressure (SBP)>140 mmHg and/or diastolic blood pressure (DBP)>90 mmHg in untreated subjects. Treated hypertensive patients were included regardless of their blood pressure (BP) values. Blood pressure was measured between 9 and 11 AM with a mercury-calibrated aneroid cuff manometer after a rest for at least 5 min in the sitting position. Three measurements were taken at one-min intervals, and the average was used to define clinical SBP and DBP. Pulse pressure (PP) was calculated as the difference between SBP and DBP.

The validation cohort consisted of participants in the Action in Diabetes and Vascular disease (ADVANCE) study, which involved a total of 11,140 patients with type 2 diabetes randomized to treatment with perindopril-indapamide or placebo.¹⁹ Genotype information was available for 1,900 patients (Table 1 below), 77% of which were hypertensive, all were Caucasians (ascertained by Structure, http://pritch.bsd.uchicago.edu/structure.html) and had Diabetes type 2 mellitus.

### Biochemistry

Serum electrolytes, fasting glucose, serum total cholesterol, triglycerides and high density lipoprotein (HDL)-cholesterol were determined by standard enzymatic colorimetric methods. Plasma catecholamine levels were determined by high-performance liquid chromatography as previously reported.¹⁸ Quantitative determination of plasma renin activity was performed with radioimmunometric assay (RIA) kit (CIS bio international, France, distributed by Bio-Rad Diagnostic, Toronto, Canada). Aldosterone was measured with RIA kit (Diagnostic Systems Laboratories Inc, Texas, USA distributed by Somagen diagnostics Inc, Edmonton, Canada). Plasma NT-proANP levels/Plasma alpha-ANP levels were measured using a sensitive radioimmuno assay (RIA) (Immunoreactive atrial natriuretic factor (IR-ANF) in human plasma. Gutkowska J, Bourassa M, Roy D, Thibault G, Garcia R, Cantin M, Genest J. Biochem Biophys Res Commun. 1985 May 16; 128(3):1350-7).

### Electrocardiography and Echocardiographic Methods

A 12-lead ECG was recorded from all patients in supine position at a paper speed of 50 mm/s. Transthoracic M-mode, two-dimensional and Doppler echocardiograms were performed with a commercially available Philips 5500™ ultrasound according to recommendations of the American Society of Echocardiography.¹⁹ The left atrial (LA) volume was calculated by the biplane method of disks or using Simpson's formula and indexed to body surface area (BSA). The LA volume was considered enlarged when LA volume/BSA exceeded 28.0 mL/m² in both sexes.¹⁹ Left ventricular (LV) mass was calculated by Devereux's formula ¹⁹ and normalized to BSA. LV hypertrophy (LVH) was considered present when LV mass/BSA exceeded 115 g/m² in men and 95 g/m² in women.¹⁹

### Genotyping

Total genomic DNA was extracted from human blood with phenol; chloroform, precipitated with ethanol and dissolved in sterile Tris EDTA (TE) buffer. Affymetrix's GeneChip™ Mapping Hind 50K assay was used according to manufacturer's protocol. The array was scanned using the Affymetrix GeneChip™ Scanner 3000 7G. The automation of the fluidics station and the scanner was supported by the Affymetrix GeneChip™ Operating Software (GCOS1.2). GeneChip™ Genotyping Analysis Software (GTYPE) was used for evaluation of the array data, generation of genotype calls and to export array reports for biostatistical analysis.

### Statistical Analyses

Statistical analyses were performed using SPSS™ (version 15.0, SPSS, Inc). Data are presented as mean±SD or frequency in percent. Differences between cases and controls were first assessed using a multivariate Hotelling-t-test and Chi-square analyses, respectively. A log transformation was used for variables with skewed distribution. Drug-group interactions were tested for all significant variables using a multivariate general linear model using AF and drug status as fixed factors. Predictors with statistically significant (P<0.05) and biologically relevant drug-group interactions were not included in the final modeling in the present analysis.

Non genetic factors (e.g., clinical factors) were fit in a stepwise forward logistic regression procedure to identify independent predictors of AF. Briefly, at each step, the predictor with the largest score statistic whose significance value was less than 0.05 was added to the model. Then, a new predictor was entered in the model and its effect on the model was evaluated by changes in −2 log-likelihood. Variables with significant changes were kept in the model. The variables left out of the analysis at the last step all had significance values larger than 0.05. Backward stepwise methods were used to check the results of the forward models. Backward methods start with a model that includes all of the predictors. At each step, the predictor that contributed the least was removed from the model, until all of the predictors in the model were significant. Variables in the final models were retained and used for joint modeling with genetic factors.

### Genome Wide Association and Final Selection of SNPs for Modeling

SNPs were filtered out if they were not in Hardy-Weinberg equilibrium (P<0.01 in controls), had a minor allele frequency (MAF)<0.10, or a call rate<95%. Cluster analysis (P-link™)²⁰ was used to identify genetic outliers. Allelic association studies and logistic regression (with covariates) were performed using P-link™.²⁰ One million simulations using the label-swapping approach as implemented in P-link™,²⁰ were conducted to calculate empirical P-values for nominal associations when P-value<1×10⁻⁴. This approach permutes the phenotype-genotype relationship but preserves the patterns of LD between SNPs under the observed and permuted samples.

Replication studies of top associated SNPs (empirical P-value≦10⁻⁵) were undertaken in a large cohort of participants of the ADVANCE study, which involved a total of 11,140 patients with type 2 diabetes randomized to treatment with perindopril-indapamide or placebo.²¹ Genotype information (Affymetrix Genome-Wide Human SNP Array 5.0) was available for 1,900 patients (Table 1 below). When discovery SNPs were not available, proxy SNPs (From HapMap-CEU 15: D′=1, LOD>2 and r2>0.6 determined by Haploview²²) were used if they were in Hardy-Weinberg equilibrium (P<0.01 in controls) or had a minor allele frequency (MAF)<0.10. Allelic association and logistic analysis with covariates was conducted using P-link™. SNPs were selected for final modeling if they were found associated with AF at P<0.01 in the replication cohort. Combined-P values were calculated using the Fisher's method as implemented in Haploview²². Replicated SNPs were used for final modeling with or without clinical and metabolic biomarkers, having the presence or absence of the risk allele as a dependent variable. Predictive probabilities of models containing only clinical, clinical and biomarkers, clinical, biomarkers and genetic factors were used to construct ROC curves.

TABLE 1
Clinical characteristics of individuals with and without
AF in the replication cohort (ADVANCE study).

| | Mean | SD | Mean | SD |
|---|---|---|---|---|
| Male sex, % | 67.4 | | 67.1 | |
| age, years | 66.7 | 6.8 | 68.9 | 6.8 |
| hba1c, % | 7.3 | 1.4 | 7.3 | 1.5 |
| BMI, kg/m2 | 29.8 | 5.0 | 29.4 | 5.1 |
| creatinine clearance | 86.8 | 28.8 | 78.2 | 29.5 |
| Hypertension, % | 76.3 | | 87.0 | |
| Previous myocardial infarction, % | 23.8 | | 43.0 | |
| Heart failure, % | 3.0 | | 20.0 | |
| Medications | | | | |
| perindopril, % | 10.0 | | 13.0 | |
| Other ACE inhibitors, % | 28.0 | | 43.0 | |
| ARBs, % | 5.0 | | 7.0 | |

Controls: Individuals without history of atrial fibrillation. Cases: Invididuals with paroxysmal Afib. All participants were Caucasian and had diabetes type 2.

### Example 2

### Results

The characteristics of participants are shown in Table 2 below. The mean age, the percentage of women and mean BMI were similar for both groups. HT+AF patients had higher waist circumference, SBP and pulse pressures compared with HT patients (Table 2). Electrocardiographic assessment showed that HT+AF patients had lower HR. However, no differences in the duration of QRS, QT and P-R were found between groups. Echocardiographic LA volume was greater in HT+AF compared with HT patients (Table 2). However, there were not differences in LV mass, interventricular or posterior wall thickness. Metabolic and neurohormonal profiling showed that HT+AF patients had higher HDL and lower fasting glucose, but a trend towards increased total cholesterol and triglyceride levels compared with HT patients (Table 2). Moreover, HT+AF patients had lower plasma ANP, aldosterone, and renin levels, and a trend towards lower plasma adrenaline without differences in noradrenaline levels (Table 2).

TABLE 2
Clinical characteristics of hypertensives with
(HT + AF) and without (HT) history of AF

| | HT | HT + AF | |
|---|---|---|---|
| | (n = 74) | (n = 74) | |
| Characteristic | Mean SD | Mean SD | P-value* |
| Age, years | 63.78 ± 9.16 | 65.11 ± 10.58 | 0.42 |
| Male, % | 60.8 | 56.0 | 0.55 |
| Body mass index, kg/m² | 27.73 ± 3.37 | 27.93 ± 4.57 | 0.76 |
| Waist circumference, cm | 94.41 ± 9.93 | 98.62 ± 11.72 | 0.02 |
| Systolic blood pressure, mmHg | 127.39 ± 13.40 | 136.55 ± 19.60 | 0.001 |
| Diastolic blood pressure, mmHg | 82.96 ± 7.52 | 81.53 ± 10.84 | 0.35 |
| Pulse pressure, mmHg | 44.43 ± 13.70 | 55.01 ± 17.37 | 0.0001 |
| Heart rate on ECG, beats/min | 61.91 ± 7.63 | 59.12 ± 8.75 | 0.04 |
| QRS, ms | 92 ± 17 | 95 ± 15 | 0.21 |
| QTc, ms | 424 ± 24 | 429 ± 24 | 0.24 |
| P-R, ms | 168.97 ± 20.28 | 174.44 ± 26.64 | 0.16 |
| LA volume/BSA, ml/m² | 31.75 ± 9.82 | 36.19 ± 11.92 | 0.02 |
| LV mass/BSA, g/m² | 94.65 ± 25.04 | 89.55 ± 24.65 | 0.21 |
| LV ejection fraction, % | 65.34 ± 4.78 | 64.73 ± 4.50 | 0.43 |
| Interventricular septum thickness, mm | 9.86 ± 1.91 | 9.45 ± 1.64 | 0.16 |
| Posterior wall thickness, mm | 9.64 ± 1.69 | 9.52 ± 1.84 | 0.67 |
| Fasting glucose, mmol/L | 5.30 ± 0.70 | 4.98 ± 0.99 | 0.03 |
| Noradrenaline, pg/ml | 306.89 ± 123.46 | 303.93 ± 145.07 | 0.89 |
| Adrenaline, pg/ml | 22.12 ± 16.40 | 18.12 ± 11.27 | 0.09 |
| Triglycerides, mmol/L | 1.43 ± 0.68 | 1.66 ± 0.87 | 0.07 |
| HDL cholesterol, mmol/L | 1.33 ± 0.33 | 1.53 ± 0.77 | 0.04 |
| Total cholesterol, mmol/L | 4.82 ± 0.78 | 5.07 ± 1.03 | 0.09 |
| ANP, fmol/ml | 225.99 ± 120.54 | 126.08 ± 113.18 | 0.000001 |
| Renin, pg/ml | 12.17 ± 19.71 | 9.27 ± 22.09 | 0.02 |
| Aldosterone, pmol/L | 317.48 ± 161.55 | 227.16 ± 74.91 | 0.00003 |
| Sodium, meq/L | 141.68 ± 1.71 | 141.88 ± 1.84 | 0.48 |
| Potassium, meq/L | 4.04 ± 0.31 | 4.16 ± 0.40 | 0.06 |

Data are expressed as mean (+/−SD) or frequency (in percent). HT (Hypertensives without history of Atrial fibrillation); HT + AF (Hypertensives with history of paroxysmal atrial fibrillation). QTc, corrected QT interval; QRS, duration of QRS complex; P-R, P-R interval, BSA, body surface area; LA, left atrial; LV. left ventricle; ANP, atrial natriuretic peptide; HDL, high-density lipoprotein.
*2 tailed P-values.

Only HT+AF patients used class I and class III antiarrhythmics (48%) or warfarin (49%). Compared with HT-AF, HT without AF used more aspirin (66 vs 32%; P<0.001), renin-angiotensin converting enzyme inhibitors (ACEIs) or angiotensin II receptor 1 blockers (ARBs) (71 vs. 43%; P<0.001). There were no significant differences in the use of beta-blockers, calcium channel blockers, diuretics and lipid-lowering drugs between both groups. Significant drug-group interactions were detected for glucose with ACE inhibitors, ARBs or statins. Likewise, significant interactions were found for aldosterone and renin with ACE inhibitors and ARBs. No drug-group interaction was found for ANP levels even when multiple drug-interactions were evaluated. Variables with significant drug-group interactions were not included in the final modeling.

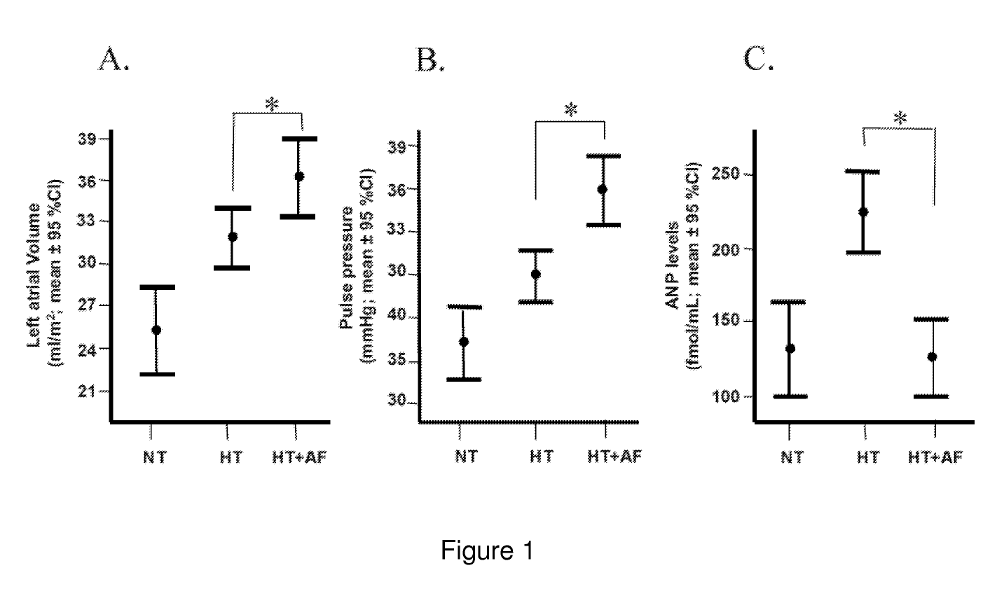

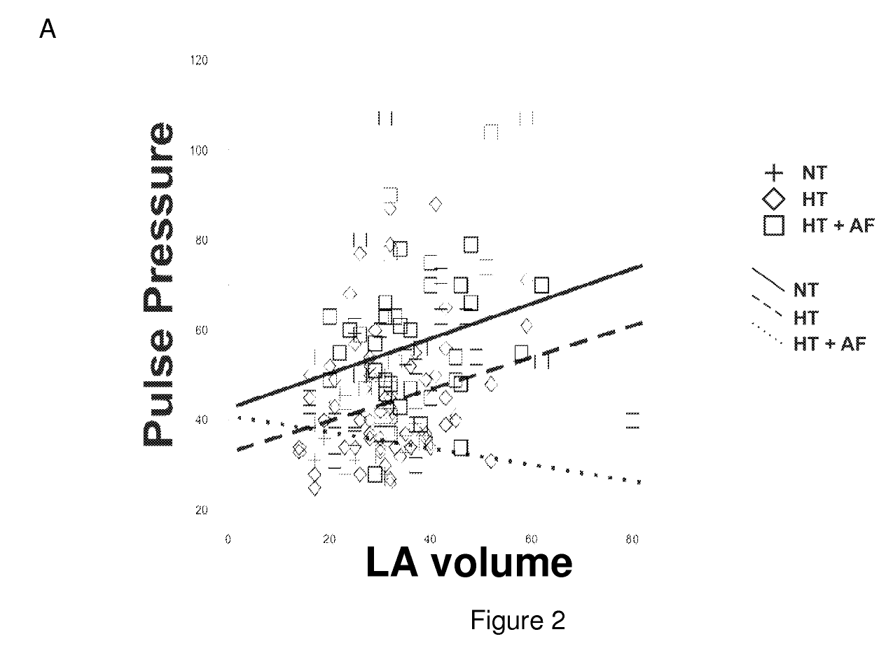

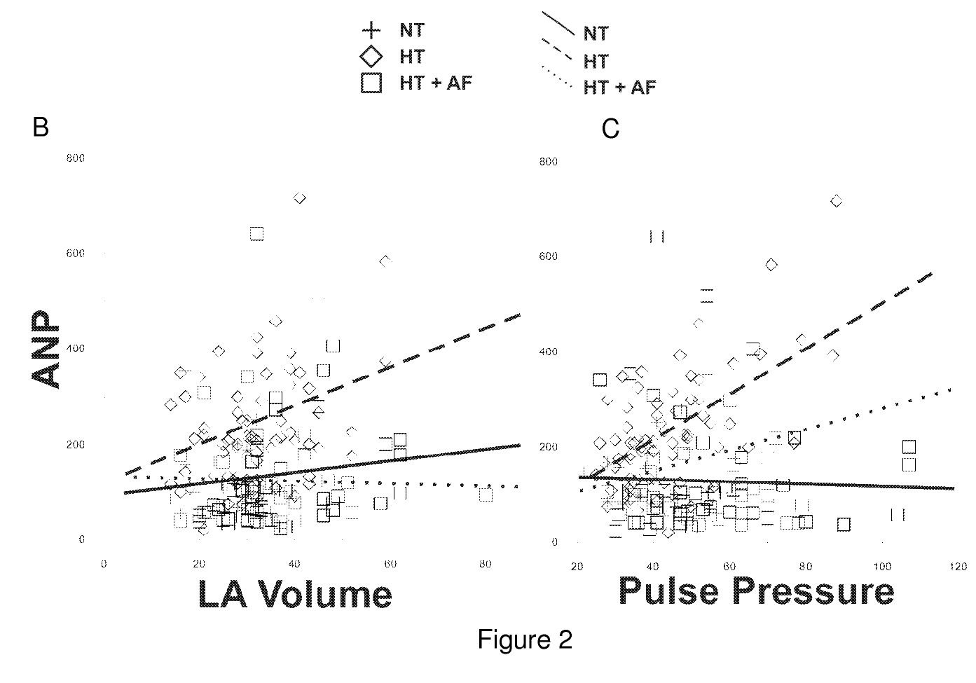

After stepwise logistic regression, two clinical factors (OR; 95% CI; top vs lowest tertile): pulse pressure (7.7; 2.6-22.9)) and LA volume (4.4; 1.1-12.1) (echocardiographic variable), and one neuroendocrine factor: ANP (0.02; 0.004-0.07) remained as independent predictors and contributed to AF risk (FIG. 1, and Table 3 below). FIG. 1 shows averages and 95% CI for main predictors of AF in hypertensive individuals with and without history of AF as well as the distribution of these factors in a group of normotensive without history of AF. Compared with normotensive (NT), hypertensive individuals (HT) had higher pulse pressure and greater LA volume compared with normotensives, these differences further increased in hypertensive that have developed AF (HT+AF) (FIG. 1). Although ANP levels were higher in hypertensive, HT+AF patients had lower ANP levels similar to that of normotensive (FIG. 1). There was a positive correlation between pulse pressure and LA volume in all hypertensive individuals, which was not seen in normotensive (FIG. 2A). ANP levels correlated with both pulse pressure and LA volume in hypertensive but this relationship was lost or decreased in those with history of AF (FIGS. 2B and 2C).

TABLE 3
Risk of AF associated with risk factors

| | Prevalence | | | | | |
|---|---|---|---|---|---|---|
| Risk factor | HT | HT + AF | Odds ratio | | Odds ratio (95% CI) | |
| (Tertiles) | (%) | (%) | (95% CI) | | multivariate adjustment | |
| LA vol (ml/m²) | | | | | | |
| <30 | 42.0 | 25.7 | | | | |
| 30-40 | 40.6 | 47.3 | 1.91* | (0.9-4.1) | 2.50 | (1.1-6.2) |
| >40 | 17.4 | 27.0 | 2.54* | (1.01-6.38) | 4.40 | (1.1-12.1) |
| Pulse P (mmHg) | | | | | | |
| <40 | 75.0 | 25.0 | | | | |
| 40-50 | 52.0 | 48.0 | 2.77* | (1.12-6.84) | 2.90 | (1.07-7.98) |
| >50 | 29.1 | 70.9 | 7.31* | (2.91-18.4) | 7.70 | (2.6-22.9) |
| ANP (fmol/ml) | | | | | | |
| <100 | 10.8 | 54.6 | | | | |
| 100-200 | 35.1 | 29.3 | 0.16* | (0.06-0.42) | 0.13 | (0.04-0.44) |
| >200 | 54 | 16 | 0.05* | (0.02-0.15) | 0.02 | (0.004-0.07) |
| rs4674485A^{†} | 18.6 | 48 | 4 | (1.9-8.6) | 2.97 | (1.12-7.83) |
| rs1466560G^{†} | 13.2 | 44 | 5.15 | (2.23-11.89) | 2.83 | (1.02-7.86) |

Table shows clinical, neuroendocrine and genetic factors associated with the development of atrial fibrillation among hypertensive individuals. Continuous traits were categorized in tertiles. Percentage of individuals and odds ratio by tertile categories in cases (HT + AF) or controls (HT) are shown. Odds ratio and 95% CI were calculated by logistic regression analysis. The lowest tertile of each continous trait or no carriers of genetic risk variants were the reference group. Estimates were calculated for models without covariates and after adjustment by clinical covariates.
*Top tertiles vs. lowest tertile.
^{†}Presence of the risk allele.

In summary, three non-genetic factors: PP, LA volume and ANP, remained as independent predictors of AF among hypertensive individuals. Hypertensives with paroxysmal AF had wider PP, larger LA and lower ANP levels. In this cohort of hypertensives, wider pulse pressure was associated with enlarged LA. However, there was a positive correlation between PP, LA volume and ANP levels only in hypertensive without history of AF, and this correlation was lost in hypertensives with history of paroxysmal AF.

First, these observations suggest that PP is a more powerful predictor of AF than systolic or diastolic BP. Second, PP could predispose to AF directly or by causing enlarged LA. Moreover, once LA is enlarged it becomes an independent risk factor (e.g., regardless of BP) for AF. Thus, in the context of hypertensive individuals without chronic AF, these observations strongly suggest that low ANP is a marker of the predisposition to develop AF, especially in those individuals with enlarged LA.

### Joint Modeling of Genetic and Clinical and Neuroendocrine Risk Factors

One million permutations were performed to calculate empirical P-values for SNPs with a nominal P-value<1×10⁻⁴. Table 4 below shows top SNPs associated with AF in this cohort of hypertensive individuals. Top empirical score was obtained for rs4674485 (P-value: 6.0×10⁻⁶). Moreover, other associated variants were found on chromosomes 2, 3, 4, 5, 7 and 10. The minor allele frequency of top risk markers ranged from 0.10 to 0.44 in this population.

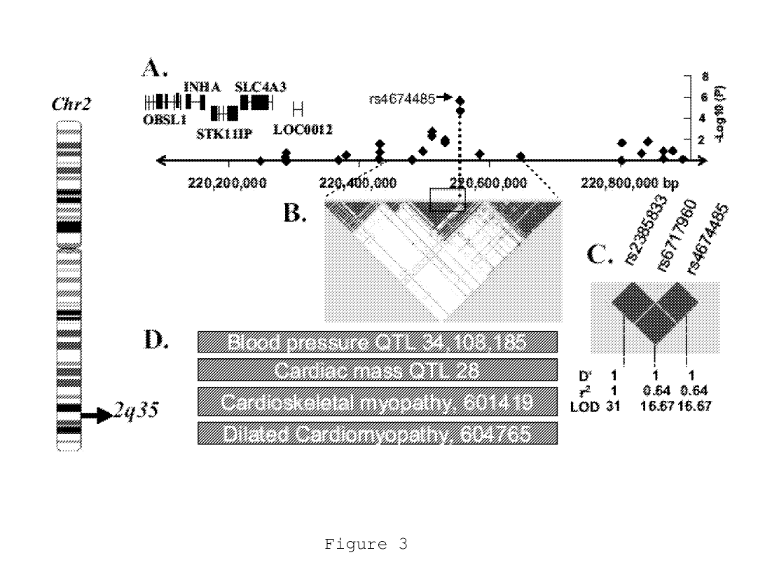

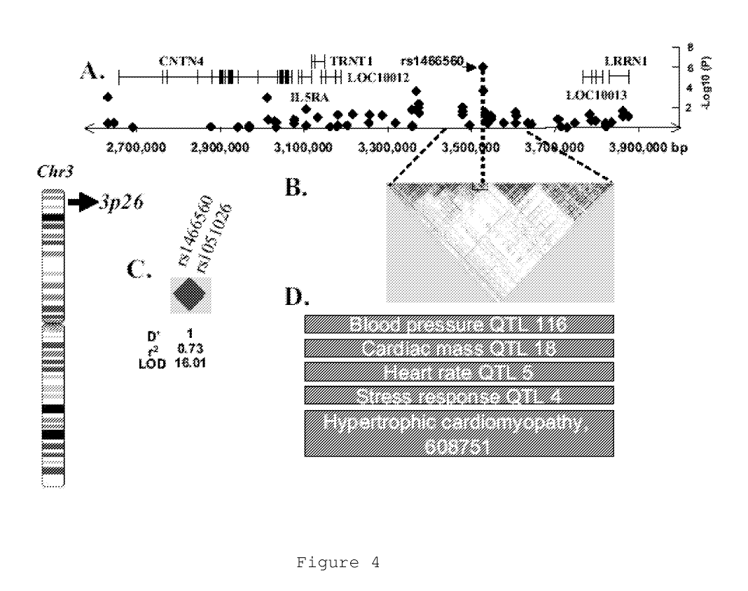

In order to select SNPs for final modeling, replication studies were conducted in a second cohort. Two SNPs (rs4674485 and rs1466560) were replicated by substitute SNPs (Table 5 below). LD maps from CEU HapMap™ population¹⁵ show that rs4674485 is in strong LD with rs6717960 (SEQ ID NO: 7) and rs2385833 (SEQ ID NO: 6) (FIG. 3, and Table 5 below), which were found associated with AF in participants of the ADVANCE Trial (P<0.01). This SNP rs4674485 [A/G] had a prevalence (0.20) in this cohort comparable to that observed in the HapMap™-CEU population (0.21)¹⁵. Moreover, hypertensive individuals with a history of paroxysmal AF had ˜3-fold higher prevalence of this allele compared with hypertensive individuals without paroxysmal AF (Table 4 below). rs4674485 is located in a genomic region where several QTLs for cardiac and vascular function have been identified (FIG. 4).

TABLE 4
Top association results for AF discovery cohort

| | | Minor | Minor Allele Frequencies | | | | |
|---|---|---|---|---|---|---|---|
| SNP | Chr | allele | HT | HT + AF | ALL* | OR (95% CI) | P value |
| rs4674485 | 2 | A | 0.10 | 0.28 | 0.20 | 3.6 (1.8, 7.1) | 6.0 × 10⁻⁶ |
| rs2299277 | 7 | T | 0.08 | 0.27 | 0.18 | 4.1 (1.9, 8.4) | 9.0 × 10⁻⁶ |
| rs2952860 | 4 | T | 0.15 | 0.35 | 0.26 | 3.0 (1.6, 5.5) | 4.2 × 10⁻⁵ |
| rs1880039 | 2 | C | 0.10 | 0.20 | 0.10 | 2.4 (1.2, 4.8) | 4.4 × 10⁻⁵ |
| rs1466560 | 3 | G | 0.07 | 0.25 | 0.17 | 4.2 (1.9, 9.1) | 4.5 × 10⁻⁵ |
| rs7039 | 3 | G | 0.57 | 0.35 | 0.44 | 0.4 (0.2, 0.6) | 5.8 × 10⁻⁵ |
| rs9312515 | 4 | G | 0.05 | 0.22 | 0.14 | 5.6 (2.3, 13.8) | 1.0 × 10⁻⁴ |
| rs3849387 | 2 | G | 0.07 | 0.20 | 0.13 | 3.7 (1.6, 8.2) | 1.2 × 10⁻⁴ |
| rs1897527 | 5 | A | 0.13 | 0.35 | 0.28 | 3.6 (1.8, 6.8) | 2.0 × 10⁻⁴ |
| rs2418828 | 10 | G | 0.32 | 0.53 | 0.41 | 2.4 (1.5, 3.9) | 2.5 × 10⁻⁴ |

Top SNPs associated with AF in hypertensives. ORs and 95% CI for the presence of the risk allele in cases (HT + AF) vs. controls (HT).
*Minor allele frequency in all hypertensive individuals. One million permutations were performed to calculate empirical P-values.

Except for rs7039, all minor alleles presented in Table 4 above are also risk alleles. For rs7039 however, the other/major allele is the risk allele. This explains the OR lower than 1 (i.e. 0.4) for the minor allele of rs7039. The risk allele for rs7039 is presented at FIG. 6.

TABLE 5
Replication studies

| Discovery cohort (n = 149) | | | | Replication cohort (n = 1900) | | | Combined |
|---|---|---|---|---|---|---|---|
| SNP id | P-value | MAF | OR (95% CI) | P-value | MAF | OR (95% CI) | P-value |
| rs4674485 | 6.0 × 10⁻⁶ | 0.19 | 3.58 (1.8-7.1) | 6.0 × 10⁻³ | 0.35 | 1.55 (1.0-2.45) | 6.5 × 10⁻⁷ |
| rs1466560 | 4.4 × 10⁻⁵ | 0.17 | 4.25 (1.9-9.1) | 2.0 × 10⁻³ | 0.20 | 2.46 (1.26-4.81) | 1.5 × 10⁻⁶ |

Top SNPs replicated in a second cohort (n = 1900) of partipants of the ADVANCE study. Proxy SNPs were used if they were in linkage disequilibrium; D′ = 1 and LOD > 2 and r > 0.6.
*rs6717960 (D′ = 1, r = 0.64; LOD = 16.67);
**rs10510266 (D′ = 1. r = 0.73; LOD = 16.01)

rs1466560 (chromosome 3) was replicated by proxy SNP rs10510266 (SEQ ID NO:11) in the ADVANCE cohort (Table 5 above). The risk variant [G] was 4 times more frequent in hypertensive with history of paroxysmal AF than in those without AF. rs1466560 is located in a genomic region where QTLs for heart rate and stress responses have been identified (FIG. 4). The prevalence of rs1466560 (0.17) in this cohort was slightly higher than that observed in the HapMap-CEU population (0.14).¹⁵

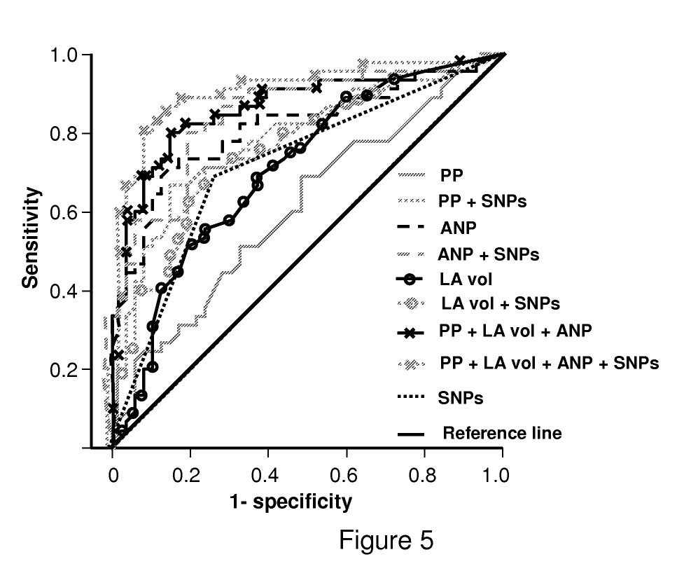

Table 3 above shows the associated risk (ORs) for individuals with higher vs. lower levels of risk factors (or presence vs. absence of risk alleles) before and after multivariate adjustment. After adjustment for other risk factors, rs4674485 or rs1466560 were associated with a ˜3-fold increase in the risk of developing AF in hypertensive individuals. ROC curves (FIG. 5) show the specificity of the models containing one or multiple factors at different levels of sensitivity. The single factor with the highest sensitivity/specificity was ANP with an area under the receiver operating characteristic curve (AUC) of 0.81 (FIG. 5; and Table 6 below). When ANP, pulse pressure and LA volume were used together, the AUC increased to 0.87 (P<1×10⁻¹³). The use of SNPs without clinical covariates had an AUC of 0.71, similar to that of atrial volume (Table 6 below). For all predictors AUC increased after adding genetic information (Table 6). The combined use of clinical, factors and genetic markers increased the AUC to 0.90 (P<1×10⁻¹⁶). ROC curves show that at sensitivity levels>60% the use of SNPs increased the specificity of the combined model (FIG. 5).

TABLE 6
Area under the curve for multiple risk factors

| Test variables | AUC | P value |
|---|---|---|
| SNPs | 0.71 | 1.5 × 10⁻⁵ |
| ANP | 0.81 | 3.1 × 10⁻¹⁰ |
| ANP + SNPs* | 0.85 | 1.5 × 10⁻¹² |
| Pulse pressure | 0.61 | 2.5 × 10⁻² |
| Pulse pressure + SNPs* | 0.79 | 6.6 × 10⁻⁹ |
| LA volume | 0.71 | 2.8 × 10⁻⁵ |
| LA volume + SNPs* | 0.76 | 1.7 × 10⁻⁷ |
| PP + LA volume + ANP | 0.87 | 1.0 × 10⁻¹³ |
| PP + LA volume + ANP + SNPs* | 0.9 | 3.6 × 10⁻¹⁶ |

*Presence of any risk allele (rs4674485A or rs 1466560G).
AUC, area under the curve from ROC curves in FIG. 4. PP: pulse pressure; LA left atrium; ANP: Atrial natriuretic peptide.

The above examples revealed SNPs (e.g., rs4674485A/G and rs1466560G/T) located on chromosomes 2 and 3 respectively), associated with a history of paroxysmal AF among hypertensive individuals. Moreover, AUC comparison showed that including these genetic markers improves the AUC of traditional risk factor models containing clinical, echocardiographic or neuroendocrine factors in a well-characterized cohort of hypertensive individuals.

### Example 3

### Characteristics of Polymorphic Markers rs4674485 and rs1466560

SNP rs4674485, the top marker associated with AF according to the present invention, is located in an area where QTLs for blood pressure,³⁰ and cardiac mass ³¹ have been identified. SLC4A3 is located ˜300 kb upstream rs4674485 (FIG. 3). This gene encodes a protein integral to plasma membrane that exchanges Cl⁻/HCO₃⁻ and contributes to regulation of intracellular pH and chloride in cardiac muscle, with possible effects on excitability and contractility.³² Human SLC4A3/AE3 polymorphisms have been associated with seizure disorders.³³ However, it is unknown whether mutations of this gene were associated with AF.

SNP rs1466560 is located in a region harboring QTLs in rodents for heart rate,³⁰ blood pressure,³⁴ cardiac mass and stress responses (corticosterone levels and stress-related disease susceptibility).³⁵ This area has also been previously identified by linkage analysis to harbor QTLs causing dilated cardiomiopathy associated with rhythm and conduction abnormalities, including atrial flutter or fibrillation.³⁶ Other neighboring genes includes tRNA nucleotidyl transferase (TRNT1), involved in RNA processing, and contactin 4 (CNTN4), a membrane protein that functions as a cell adhesion molecule and may play a role in the formation of axon connections in the developing nervous system.³⁷

Although the present invention has been described hereinabove by way of specific embodiments thereof, it can be modified, without departing from the spirit and nature of the subject invention as defined in the appended claims.

### REFERENCES

1. Miyasaka Y, Barnes M E, Gersh B J, Cha S S, Bailey K R, Abhayaratna W P, Seward J B, Tsang TS. Secular trends in incidence of atrial fibrillation in Olmsted County, Minnesota, 1980 to 2000, and implications on the projections for future prevalence. Circulation. 2006; 114:119-125.
2. Hart R G, Benavente O, McBride R, Pearce L A. Antithrombotic therapy to prevent stroke in patients with atrial fibrillation: a meta-analysis. Ann Intern Med. 1999; 131:492-501.
3. Benjamin E J, Wolf P A, D'Agostino R B, Silbershatz H, Kannel W B, Levy D. Impact of atrial fibrillation on the risk of death: the Framingham Heart Study. Circulation. 1998; 98:946-952.
4. Du X, Ninomiya T, de Galan B, Abadir E, Chalmers J, Pillai A, Woodward M, Cooper M, Harrap S, Hamet P, Poulter N, Lip G Y, Patel A. Risks of cardiovascular events and effects of routine blood pressure lowering among patients with type 2 diabetes and atrial fibrillation: results of the ADVANCE study. Eur Heart J. 2009.
5. Ellinor P T, Yi B A, MacRae C A. Genetics of atrial fibrillation. Med Clin North Am. 2008; 92:41-51, x.
6. Brugada R. Is atrial fibrillation a genetic disease? J Cardiovasc Electrophysiol. 2005; 16:553-556.
7. Fox C S, Parise H, D'Agostino R B, Sr., Lloyd-Jones D M, Vasan R S, Wang T J, Levy D, Wolf P A, Benjamin E J. Parental atrial fibrillation as a risk factor for atrial fibrillation in offspring. Jama. 2004; 291:2851-2855.
8. Chen Y H, Xu S J, Bendahhou S, Wang X L, Wang Y, Xu W Y, Jin H W, Sun H, Su X Y, Zhuang Q N, Yang Y Q, Li Y B, Liu Y, Xu H J, Li X F, Ma N, Mou C P, Chen Z, Barhanin J, Huang W. KCNQ1 gain-of-function mutation in familial atrial fibrillation. Science. 2003; 299:251-254.
9. Yang Y, Xia M, Jin Q, Bendahhou S, Shi J, Chen Y, Liang B, Lin J, Liu Y, Liu B, Zhou Q, Zhang D, Wang R, Ma N, Su X, Niu K, Pei Y, Xu W, Chen Z, Wan H, Cui J, Barhanin J, Chen Y. Identification of a KCNE2 gain-of-function mutation in patients with familial atrial fibrillation. Am J Hum Genet. 2004; 75:899-905.
10. Brugada R, Tapscott T, Czernuszewicz G Z, Marian A J, Iglesias A, Mont L, Brugada J, Girona J, Domingo A, Bachinski L L, Roberts R. Identification of a genetic locus for familial atrial fibrillation. N Engl J Med. 1997; 336:905-911.
11. Ellinor P T, Shin J T, Moore R K, Yoerger D M, MacRae C A. Locus for atrial fibrillation maps to chromosome 6q14-16. Circulation. 2003; 107:2880-2883.
12. Arnar D O, Thorvaldsson S, Manolio T A, Thorgeirsson G, Kristjansson K, Hakonarson H, Stefansson K. Familial aggregation of atrial fibrillation in Iceland. Eur Heart J. 2006; 27:708-712.
13. Gudbjartsson D F, Amar D O, Helgadottir A, Gretarsdottir S, Holm H, Sigurdsson A, Jonasdottir A, Baker A, Thorleifsson G, Kristjansson K, Palsson A, Blondal T, Sulem P, Backman V M, Hardarson G A, Palsdottir E, Helgason A, Sigurjonsdottir R, Sverrisson J T, Kostulas K, Ng M C, Baum L, So W Y, Wong K S, Chan J C, Furie K L, Greenberg S M, Sale M, Kelly P, MacRae C A, Smith E E, Rosand J, Hillert J, Ma R C, Ellinor P T, Thorgeirsson G, Gulcher J R, Kong A, Thorsteinsdottir U, Stefansson K. Variants conferring risk of atrial fibrillation on chromosome 4q25. Nature. 2007; 448:353-357.
14. Larson M G, Atwood L D, Benjamin E J, Cupples L A, D'Agostino R B, Sr., Fox C S, Govindaraju D R, Guo C Y, Heard-Costa N L, Hwang S J, Murabito J M, Newton-Cheh C, O'Donnell C J, Seshadri S, Vasan R S, Wang T J, Wolf P A, Levy D. Framingham Heart Study 100K project: genome-wide associations for cardiovascular disease outcomes. BMC Med Genet. 2007; 8 Suppl 1:S5.
15. The International HapMap Consortium. A haplotype map of the human genome. Nature. 2005; 437:1299-1320.
16. Kannel W B, Wolf P A, Benjamin E J, Levy D. Prevalence, incidence, prognosis, and predisposing conditions for atrial fibrillation: population-based estimates. Am J Cardiol. 1998; 82:2 N-9N.
17. Dahlof B, Devereux R B, Kjeldsen S E, Julius S, Beevers G, de Faire U, Fyhrquist F, Ibsen H, Kristiansson K, Lederballe-Pedersen O, Lindholm L H, Nieminen M S, Omvik P, Oparil S, Wedel H. Cardiovascular morbidity and mortality in the Losartan Intervention For Endpoint reduction in hypertension study (LIFE): a randomised trial against atenolol. Lancet. 2002; 359:995-1003.
18. De Champlain J, Karas M, Nguyen P, Cartier P, Wistaff R, Toal C B, Nadeau R, Larochelle P. Different effects of nifedipine and amlodipine on circulating catecholamine levels in essential hypertensive patients. J Hypertens. 1998; 16:1357-1369.
19. Lang R M, Bierig M, Devereux R B, Flachskampf F A, Foster E, Pellikka P A, Picard M H, Roman M J, Seward J, Shanewise J S, Solomon S D, Spencer K T, Sutton M S, Stewart W J. Recommendations for chamber quantification: a report from the American Society of Echocardiography's Guidelines and Standards Committee and the Chamber Quantification Writing Group, developed in conjunction with the European Association of Echocardiography, a branch of the European Society of Cardiology. J Am Soc Echocardiogr. 2005; 18:1440-1463.
20. Purcell S, Neale B, Todd-Brown K, Thomas L, Ferreira M A, Bender D, Mailer J, Sklar P, de Bakker P I, Daly M J, Sham PC. PLINK: a tool set for whole-genome association and population-based linkage analyses. Am J Hum Genet. 2007; 81:559-575.
21. Patel A, MacMahon S, Chalmers J, Neal B, Billot L, Woodward M, Marre M, Cooper M, Glasziou P, Grobbee D, Hamet P, Harrap S, Heller S, Liu L, Mancia G, Mogensen C E, Pan C, Poulter N, Rodgers A, Williams B, Bompoint S, de Galan B E, Joshi R, Travert F. Intensive blood glucose control and vascular outcomes in patients with type 2 diabetes. N Engl J Med. 2008; 358:2560-2572.
22. Barrett J C, Fry B, Mailer J, Daly M J. Haploview: analysis and visualization of LD and haplotype maps. Bioinformatics. 2005; 21:263-265.
23. Aronow W S. Etiology, pathophysiology, and treatment of atrial fibrillation: part 1. Cardiol Rev. 2008; 16:181-188.
24. Savelieva I, Camm A J. Clinical relevance of silent atrial fibrillation: prevalence, prognosis, quality of life, and management. J Intery Card Electrophysiol. 2000; 4:369-382.
25. Becker R C. Biomarkers in atrial fibrillation: investigating biologic plausibility, cause, and effect. J Thromb Thrombolysis. 2005; 19:71-75.
26. Issac T T, Dokainish H, Lakkis N M. Role of inflammation in initiation and perpetuation of atrial fibrillation: a systematic review of the published data. J Am Coll Cardiol. 2007; 50:2021-2028.
27. Roldan V, Marin F, Lip G Y. Letter by Roldan et al regarding article, “Plasminogen activator inhibitor-1 as a predictor of postoperative atrial fibrillation after cardiopulmonary bypass”. Circulation. 2008; 117:e169.
28. Burke M A, Cotts W G. Interpretation of B-type natriuretic peptide in cardiac disease and other comorbid conditions. Heart Fail Rev. 2007; 12:23-36.
29. Patel M J, de Lemos J A. Can biomarkers improve prediction of future cardiovascular events in patients with cardiovascular disease? Nat Clin Pract Cardiovasc Med. 2007; 4:14-15.
30. Moreno C, Dumas P, Kaldunski M L, Tonellato P J, Greene A S, Roman R J, Cheng O, Wang Z, Jacob H J, Cowley A W, Jr. Genomic map of cardiovascular phenotypes of hypertension in female Dahl S rats. Physiol Genomics. 2003; 15:243-257.
31. Garrett M R, Saad Y, Dene H, Rapp J P. Blood pressure QTL that differentiate Dahl saltsensitive and spontaneously hypertensive rats. Physiol Genomics. 2000; 3:33-38.
32. Yannoukakos D, Stuart-Tilley A, Fernandez H A, Fey P, Duyk G, Alper S L. Molecular cloning, expression, and chromosomal localization of two isoforms of the AE3 anion exchanger from human heart. Circ Res. 1994; 75:603-614.
33. Alper S L. Molecular physiology of SLC4 anion exchangers. Exp Physiol. 2006; 91:153-161.
34. Garrett M R, Joe B, Dene H, Rapp J P. Identification of blood pressure quantitative trait loci that differentiate two hypertensive strains. J Hypertens. 2002; 20:2399-2406.
35. Potenza M N, Brodkin E S, Joe B, Luo X, Remmers E F, Wilder R L, Nestler E J, Gelernter J. Genomic regions controlling corticosterone levels in rats. Biol Psychiatry. 2004; 55:634-641.
36. Olson T M, Keating M T. Mapping a cardiomyopathy locus to chromosome 3p22-p25. J Clin Invest. 1996; 97:528-532.
37. Miura S, Shibata H, Furuya H, Ohyagi Y, Osoegawa M, Miyoshi Y, Matsunaga H, Shibata A, Matsumoto N, Iwaki A, Taniwaki T, Kikuchi H, Kira J, Fukumaki Y. The contactin 4 gene locus at 3p26 is a candidate gene of SCA16. Neurology. 2006; 67:1236-1241.
38. Mitchell G F, Vasan R S, Keyes M J, Parise H, Wang T J, Larson M G, D'Agostino R B, Sr., Kannel W B, Levy D, Benjamin E J. Pulse pressure and risk of new-onset atrial fibrillation. Jama. 2007; 297:709-715.
39. Thomas M C, Dublin S, Kaplan R C, Glazer N L, Lumley T, Longstreth W T, Jr., Smith N L, Psaty B M, Siscovick D S, Heckbert S R. Blood pressure control and risk of incident atrial fibrillation. Am J Hypertens. 2008; 21:1111-1116.
40. Tsang T S, Abhayaratna W P, Barnes M E, Miyasaka Y, Gersh B J, Bailey K R, Cha S S, Seward J B. Prediction of cardiovascular outcomes with left atrial size: is volume superior to area or diameter? J Am Coll Cardiol. 2006; 47:1018-1023.
41. Tsang T S, Barnes M E, Bailey K R, Leibson C L, Montgomery S C, Takemoto Y, Diamond P M, Marra M A, Gersh B J, Wiebers D O, Petty G W, Seward J B. Left atrial volume: important risk marker of incident atrial fibrillation in 1655 older men and women. Mayo Clin Proc. 2001; 76:467-475.
42. Vaziri S M, Larson M G, Benjamin E J, Levy D. Echocardiographic predictors of nonrheumatic atrial fibrillation. The Framingham Heart Study. Circulation. 1994; 89:724-730.
43. Casaclang-Verzosa G, Gersh B J, Tsang T S. Structural and functional remodeling of the left atrium: clinical and therapeutic implications for atrial fibrillation. J Am Coll Cardiol. 2008; 51:1-11.
44. Jibrini M B, Molnar J, Arora R R. Prevention of atrial fibrillation by way of abrogation of the renin-angiotensin system: a systematic review and meta-analysis. Am J Ther. 2008; 15:36-43.
45. Nagata M, Hiroe M, Naruse M, Yu Z X, Sekiguchi M, Hirosawa K, Koyanagi H, Hashimoto A, Endo M, Shizume K. An integrated study employing histopathological, immunohistocytochemical and radioimmunoassay analyses of atrial natriuretic peptide in the right and left atria in patients with mitral valve disease. Jpn Circ J. 1988; 52:1453-1456.
46. van den Berg M P, van Gelder I C, van Veldhuisen D J. Depletion of atrial natriuretic peptide during longstanding atrial fibrillation. Europace. 2004; 6:433-437.
47. Risch and Merikangas, Science 273: 1516-1517 (1996).
48. Maniatis, Methods Mol Biol. 376: 109-21 (2007).
49. Borecki et al., Adv Genet 60: 51-74 (2008).
50. Maniatis, N., et al., Proc Natl Acad Sci USA 99:2228-2233 (2002)
51. Reich, D E et al, Nature 411:199-204 (2001).
52. Wall., J. D. and Pritchard, J. K., Nature Reviews Genetics 4:587-597 (2003).
53. Daly, M. et al., Nature Genet. 29:229-232 (2001).
54. Gabriel, S. B. et al., Science 296:2225-2229 (2002).
55. Patil, N. et al., Science 294: 1719-1723 (2001).
56. Dawson, E. et al., Nature 418:544-548 (2002).
57. Phillips, M. S. et al., Nature Genet 33:382-387 (2003).
58. Myers, S., et al., Science 310:321-32324 (2005).
59. Myers, S. et al., Biochem Soc Trans 34:526530 (2006).60. Gutkowska J et al. Immunoreactive atrial natriuretic factor (IR-ANF) in human plasma Biochem Biophys Res Commun. 1985 May 16; 128(3):1350-7 (1985).

1. A method of determining a predisposition to atrial fibrillation (AF) in a subject comprising: determining the presence of at least one copy of a risk allele from at least one polymorphic marker in a sample from the subject, wherein the presence of at least one copy of the risk allele is indicative of a predisposition to AF, and wherein said at least one polymorphic marker is:
a) rs4674485;
b) rs1466560;
c) rs1880039;
d) rs3849387;
e) rs7039;
f) rs2952860;
g) rs9312515;
h) rs1897527;
i) rs2299277;
j) rs2418828;
k) rs2385833;
l) rs6717960;
m) rs10510266; or
n) a substitute polymorphic marker in linkage disequilibrium with any one of the polymorphic markers of a) to m).

2. The method of claim 1, comprising comparing a sequence of said at least one polymorphic marker from the subject to a reference sequence not associated with AF.

3. The method of claim 1, wherein said sample is a nucleic acid sample.

4. The method of claim 1, wherein said sample is a protein sample.

5. The method of claim 3, wherein the reference sequence is a nucleic acid sequence comprising the sequence of: a) SEQ ID NO:2 for the polymorphic marker rs4674485; b) SEQ ID NO:4 for the polymorphic marker rs1466560; c) SEQ ID NO:14 for the polymorphic marker rs1880039; d) SEQ ID NO:15 for the polymorphic marker rs3849387; e) SEQ ID NO:10 for the polymorphic marker rs7039; f) SEQ ID NO:17 for the polymorphic marker rs2952860; g) SEQ ID NO:19 for the polymorphic marker rs9312515; h) SEQ ID NO:22 for the polymorphic marker rs1897527; i) SEQ ID NO:23 for the polymorphic marker rs2299277; j) SEQ ID NO:25 for the polymorphic marker rs2418828; k) SEQ ID NO:6 for the polymorphic marker rs2385833; l) SEQ ID NO:7 for the polymorphic marker rs6717960; m) SEQ ID NO:11 for the polymorphic marker rs10510266; or n) a complement of any one of a) to m).

6. The method of claim 1, wherein the risk allele comprises the nucleic acid sequence of: a) SEQ ID NO:27 for the polymorphic marker rs4674485; b) SEQ ID NO:28 for the polymorphic marker rs1466560; c) SEQ ID NO:33 for the polymorphic marker rs1880039; d) SEQ ID NO:34 for the polymorphic marker rs3849387; e) SEQ ID NO:31 for the polymorphic marker rs7039; f) SEQ ID NO:32 for the polymorphic marker rs2952860; g) SEQ ID NO:37 for the polymorphic marker rs9312515; h) SEQ ID NO:38 for the polymorphic marker rs1897527; i) SEQ ID NO:35 for the polymorphic marker rs2299277; j) SEQ ID NO:36 for the polymorphic marker rs2418828; k) SEQ ID NO:29 for the polymorphic marker rs2385833; l) SEQ ID NO:30 for the polymorphic marker rs6717960; m) SEQ ID NO:39 for the polymorphic marker rs10510266; or n) a complement of any one of a) to m).

7. The method of claim 6, wherein the risk allele comprises the nucleic acid sequence of: a) SEQ ID NO:1 for the polymorphic marker rs4674485; b) SEQ ID NO:3 for the polymorphic marker rs1466560; c) SEQ ID NO:13 for the polymorphic marker rs1880039; d) SEQ ID NO:16 for the polymorphic marker rs3849387; e) SEQ ID NO:9 for the polymorphic marker rs7039; f) SEQ ID NO:18 for the polymorphic marker rs2952860; g) SEQ ID NO:20 for the polymorphic marker rs9312515; h) SEQ ID NO:21 for the polymorphic marker rs1897527; i) SEQ ID NO:24 for the polymorphic marker rs2299277; j) SEQ ID NO:26 for the polymorphic marker rs2418828; k) SEQ ID NO:5 for the polymorphic marker rs2385833; l) SEQ ID NO:8 for the polymorphic marker rs6717960; m) SEQ ID NO:12 for the polymorphic marker rs10510266; or n) a complement of any one of a) to m).

8. The method of claim 1, wherein said at least one polymorphic marker comprises rs4674485.

9. The method of claim 1, wherein said at least one polymorphic marker comprises rs1466560.

10. The method of claim 1, wherein said at least one polymorphic marker is at least one substitute polymorphic marker in linkage disequilibrium with any one of the polymorphic markers of a) to j).

11. The method of claim 10, wherein said at least one substitute polymorphic marker is rs2385833 or rs6717960 and wherein rs2385833 or rs6717960 are in linkage disequilibrium with rs4674485.

12. The method of claim 10, wherein said at least one substitute polymorphic marker is rs10510266, and wherein rs10510266 is in linkage disequilibrium with rs1466560.

13. The method of claim 1, further comprising determining the presence of at least one of:
(a) pulse pressure;
(b) left atrial volume; and
(c) atrial natriuretic peptide (ANP) concentration,
wherein a higher pulse pressure and/or left atrial volume as compared to normotensive subjects or a decrease in ANP concentration as compared to hypertensive individuals is indicative of an increased risk of suffering from atrial fibrillation.

14. The method of claim 13, wherein said decrease in ANP concentration is equivalent to the ANP concentration in normotensive individuals.

15. A method of determining a predisposition to atrial fibrillation (AF) in a subject suffering from hypertension comprising:
determining at least one of
a) pulse pressure;
b) left atrial volume; and
c) Atrial Natriuretic Peptide (ANP) concentration,
in said subject,
wherein a higher pulse pressure and/or higher left atrial volume and/or a lower concentration of ANP as compared to an hypertensive subject not suffering from AF is indicative of an increased risk of suffering from AF.

16. The method of claim 13, wherein at least two of pulse pressure, left atrial volume and ANP blood concentration are determined.

17. The method of 16, wherein pulse pressure, left atrial volume and ANP blood concentration are determined.

18. The method of claim 1, wherein said AF is paroxysmal AF.

19. The method of claim 1, wherein said subject suffers from hypertension.

20. The method of claim 1, wherein said method has a specificity of at least 80%.

21. The method of claim 1, wherein said method has a specificity of at least 85%.

22. The method of claim 1, further comprising determining whether said subject is in need of preventive medication.

23. The method of claim 1, further comprising assessing the subject's probability of response to a therapeutic agent for preventing and/or ameliorating symptoms associated with AF and/or stroke.

24. A kit for determining a predisposition to atrial fibrillation (AF) in a subject comprising: reagents for determining the presence of at least one copy of a risk allele from at least one polymorphic marker in a sample from the subject, wherein the presence of at least one copy of the risk allele is indicative of a predisposition to AF and wherein said at least one polymorphic marker is:
a) rs4674485;
b) rs1466560;
c) rs1880039;
d) rs3849387;
e) rs7039;
f) rs2952860;
g) rs9312515;
h) rs1897527;
i) rs2299277;
j) rs2418828;
k) rs2385833;
l) rs6717960;
m) rs10510266; or
n) a substitute polymorphic marker in linkage disequilibrium with any one of the polymorphic markers of a) to m).

25. The kit of claim 24, further comprising reagents for determining the presence of a reference sequence of the polymorphic maker, wherein said reference sequence is not associated with AF.

26. The kit of claim 24, wherein said sample is a nucleic acid sample.

27. The kit of claim 24, wherein said sample is a protein sample.

28. The kit of claim 25, wherein the reference sequence is a nucleic acid sequence comprising the sequence of: a) SEQ ID NO:2 for the polymorphic marker rs4674485; b) SEQ ID NO:4 for the polymorphic marker rs1466560; c) SEQ ID NO:14 for the polymorphic marker rs1880039; d) SEQ ID NO:15 for the polymorphic marker rs3849387; e) SEQ ID NO:10 for the polymorphic marker rs7039; f) SEQ ID NO:17 for the polymorphic marker rs2952860; g) SEQ ID NO:19 for the polymorphic marker rs9312515; h) SEQ ID NO:22 for the polymorphic marker rs1897527; i) SEQ ID NO:23 for the polymorphic marker rs2299277; j) SEQ ID NO:25 for the polymorphic marker rs2418828; k) SEQ ID NO:6 for the polymorphic marker rs2385833; l) SEQ ID NO:7 for the polymorphic marker rs6717960; m) SEQ ID NO:11 for the polymorphic marker rs10510266; or n) a complement of any one of a) to m).

29. The kit of claim 24, wherein the risk allele comprises the nucleic acid sequence of: a) SEQ ID NO:27 for the polymorphic marker rs4674485; b) SEQ ID NO:28 for the polymorphic marker rs1466560; c) SEQ ID NO:33 for the polymorphic marker rs1880039; d) SEQ ID NO:34 for the polymorphic marker rs3849387; e) SEQ ID NO:31 for the polymorphic marker rs7039; f) SEQ ID NO:32 for the polymorphic marker rs2952860; g) SEQ ID NO:37 for the polymorphic marker rs9312515; h) SEQ ID NO:38 for the polymorphic marker rs1897527; i) SEQ ID NO:35 for the polymorphic marker rs2299277; j) SEQ ID NO:36 for the polymorphic marker rs2418828; k) SEQ ID NO:29 for the polymorphic marker rs2385833; l) SEQ ID NO:30 for the polymorphic marker rs6717960; m) SEQ ID NO:39 for the polymorphic marker rs10510266; or n) a complement of any one of a) to m).

30. The kit of claim 29, wherein the risk allele comprises the nucleic acid sequence of: a) SEQ ID NO:1 for the polymorphic marker rs4674485; b) SEQ ID NO:3 for the polymorphic marker rs1466560; c) SEQ ID NO:13 for the polymorphic marker rs1880039; d) SEQ ID NO:16 for the polymorphic marker rs3849387; e) SEQ ID NO:9 for the polymorphic marker rs7039; f) SEQ ID NO:18 for the polymorphic marker rs2952860; g) SEQ ID NO:20 for the polymorphic marker rs9312515; h) SEQ ID NO:21 for the polymorphic marker rs1897527; i) SEQ ID NO:24 for the polymorphic marker rs2299277; j) SEQ ID NO:26 for the polymorphic marker rs2418828; k) SEQ ID NO:5 for the polymorphic marker rs2385833; l) SEQ ID NO:8 for the polymorphic marker rs6717960; m) SEQ ID NO:12 for the polymorphic marker rs10510266; or n) a complement of any one of a) to m).

31. The kit of claim 24, wherein said kit comprises reagents for determining the presence of the polymorphic marker rs4674485.

32. The kit of claim 24, wherein said kit comprises reagents for determining the presence of the polymorphic marker rs1466560.

33. The kit of claim 24, wherein said kit comprises reagents for determining the presence of at least one substitute polymorphic marker in linkage disequilibrium with any one of the polymorphic markers of a) to j).

34. The kit of claim 33, wherein said at least one substitute polymorphic marker is rs2385833 or rs6717960 and wherein rs2385833 or rs6717960 are in linkage disequilibrium with rs4674485.

35. The kit of claim 33, wherein said at least one substitute polymorphic marker is rs10510266, and wherein rs10510266 is in linkage disequilibrium with rs1466560.

36. The kit of claim 24, further comprising reagents and/or instructions for determining the presence of at least one of:
(a) pulse pressure;
(b) left atrial volume; and
(c) atrial natriuretic peptide (ANP) concentration,
in said subject.

37. The kit of claim 36, wherein said decrease in ANP concentration is equivalent to the ANP concentration in normotensive individuals.

38. A kit for determining a predisposition to atrial fibrillation (AF) in a subject suffering from hypertension comprising reagents and/or instructions for determining at least one of:
(a) pulse pressure;
(b) left atrial volume; and
(c) atrial natriuretic peptide (ANP) concentration,
in said subject.

39. The kit of claim 36, comprising reagents and/or instructions for determining at least two of pulse pressure, left atrial volume and ANP blood concentration.

40. The kit of claim 39, comprising reagents and/or instructions for determining pulse pressure, left atrial volume and ANP blood concentration.

41. The kit of claim 24, for determining a predisposition to paroxysmal AF.

42. The kit of claim 24, for determining a predisposition to paroxysmal AF in a subject suffering from hypertension.

43. The kit of claim 24, wherein said reagents comprises at least one primer and/or probe.
